(19) United States
(12) Patent Application Publication
(10) Pub. No.: US 2025/0194985 A1
(43) Pub. Date: Jun. 19, 2025

(54) REMOTE ARRHYTHMIA DETECTION AND TREATMENT ANALYSIS IN WEARABLE CARDIAC DEVICES

(71) Applicant: ZOLL Medical Corporation, Chelmsford, MA (US)

(72) Inventors: Gary A. Freeman, Waltham, MA (US); Joshua Van Der Lee, Mars, PA (US); Shane S. Volpe, Saltsburg, PA (US)

(73) Assignee: ZOLL Medical Corporation, Chelmsford, MA (US)

(21) Appl. No.: 18/849,671

(22) PCT Filed: Mar. 24, 2023
(86) PCT No.: PCT/US2023/016251

§ 371 Date: Sep. 23, 2024

Related U.S. Application Data
(60) Provisional application No. 63/362,010, filed on Mar. 28, 2022.

(51) Int. Cl. A61B 5/361; A61B 5/00; A61B 5/256; A61B 5/282; A61N 1/39

(52) U.S. Cl. CPC A61B 5/361; A61B 5/0006; A61B 5/256; A61B 5/282; A61B 5/4836; A61B 5/746; A61N 1/3904

(57) ABSTRACT

A wearable cardiac monitoring and treatment system configured to remotely identify and/or verify treatable and/or alertable cardiac arrhythmias in an ambulatory patient is provided. The system includes a wearable cardiac monitoring and treatment device including ECG electrodes, therapy electrodes, and a medical device controller. The system also includes a remote server in electronic communication with the controller. The controller includes one or more processors configured to generate an ECG signal, determine one or more ECG segments corresponding to a suspected arrhythmia, transmit the ECG segment(s) to the remote server, receive, from the remote server, a remote indication as to whether the patient is experiencing an arrhythmia, independently analyze, at the medical device controller, the ECG segment(s) to generate a local indication as to whether the patient is experiencing an arrhythmia, and determine whether to deliver a therapeutic shock to the patient based on the remote and local indications.

### CROSS-REFERENCE TO RELATED APPLICATION

This nonprovisional application claims priority to U.S. Provisional Patent Application Ser. No. 63/362,010, filed on Mar. 28, 2022, titled “REMOTE ARRHYTHMIA DETECTION AND TREATMENT ANALYSIS IN WEARABLE CARDIAC DEVICES,” the entirety of which is hereby incorporated by reference.

### BACKGROUND

The present disclosure relates to a wearable cardiac monitoring and treatment system configured to monitor, manage, and/or treat patient arrhythmias.

Heart failure, if left untreated, can lead to certain life-threatening arrhythmias. Both atrial and ventricular arrhythmias are common in patients with heart failure. One of the deadliest cardiac arrhythmias is ventricular fibrillation (VF), which occurs when normal, regular electrical impulses are replaced by irregular and rapid impulses, causing the heart muscle to stop normal contractions. Because the victim has no perceptible warning of the impending fibrillation, death often occurs before the necessary medical assistance can arrive. Other cardiac arrhythmias can include excessively slow heart rates known as bradycardia or excessively fast heart rates known as tachycardia. Cardiac arrest can occur when a patient in which various arrhythmias of the heart, such as ventricular fibrillation, ventricular tachycardia (VT), pulseless electrical activity (PEA), and asystole (heart stops all electrical activity), result in the heart providing insufficient levels of blood flow to the brain and other vital organs for the support of life. It is generally useful to monitor heart failure patients to assess heart failure symptoms early and provide interventional therapies as soon as possible.

Patients may be prescribed to wear cardiac monitoring and/or cardiac treatment devices for extended periods of time. Cardiac monitoring and treatment devices may monitor the patient's heart activity and may provide defibrillation shocks to the patient if an abnormal cardiac rhythm is detected. As such, these monitoring and treatment device need to be able to identify abnormal cardiac rhythms.

### SUMMARY

In one or more examples, a wearable cardiac monitoring and treatment system configured to remotely identify and/or verify treatable and/or alertable cardiac arrhythmias in an ambulatory patient is provided. The system includes a wearable cardiac monitoring and treatment device configured for long-term continuous cardiac monitoring of the ambulatory patient. The device includes a plurality of ECG electrodes configured to be in long-term continuous contact with skin of the ambulatory patient and sense electrical signals indicative of cardiac activity in the ambulatory patient, a plurality of therapy electrodes configured to deliver one or more therapeutic electrical shocks to the ambulatory patient, and a medical device controller coupled to the plurality of ECG electrodes and/or the plurality of therapy electrodes. The system also includes a remote server in electronic communication with the medical device controller of the wearable cardiac monitoring and treatment device.

The medical device controller includes one or more processors configured to generate an ECG signal based on the sensed electrical signals indicative of the cardiac activity in the ambulatory patient, determine one or more ECG segments corresponding to a suspected arrhythmia occurring in the ambulatory patient based on the ECG signal, transmit the determined one or more ECG segments corresponding to the suspected arrhythmia occurring in the ambulatory patient to the remote server, receive, from the remote server, a remote indication based on the one or more ECG segments as to whether the ambulatory patient is experiencing a treatable and/or alertable arrhythmia, independently analyze, at the medical device controller, the one or more ECG segments to generate a local indication as to whether the ambulatory patient is experiencing a treatable and/or alertable arrhythmia, and determine whether to deliver, via the plurality of therapy electrodes, a therapeutic shock to the ambulatory patient based on the received remote indication and the local indication as to whether the ambulatory patient is experiencing a treatable and/or alertable arrhythmia.

Implementations of the wearable cardiac monitoring and treatment system can include one or more of the following features. The wearable cardiac monitoring and treatment device further includes a garment configured to be worn about a torso of the ambulatory patient. The plurality of ECG electrodes and the plurality of therapy electrodes are configured to be disposed on the garment. The one or more processors are configured to wait a predetermined amount of time for the remote indication upon generating a local indication that the ambulatory patient is experiencing a treatable and/or alertable arrhythmia. The system further includes a gateway device, and the medical device controller is at least partially implemented in the gateway device. The one or more processors are further configured to query the remote server for the remote indication upon generating a local indication that the ambulatory patient is experiencing a treatable and/or alertable arrhythmia. The query to the remote server for the remote indication includes the one or more ECG segments that are transmitted to the remote server.

The one or more processors are configured to determine whether to deliver the therapeutic shock to the ambulatory patient by determining whether the remote indication and the local indication indicate that the ambulatory patient is experiencing a treatable and/or alertable arrhythmia. The one or more processors are further configured to initiate a treatment sequence upon determining that both the remote indication and the local indication indicate that the ambulatory patient is experiencing a treatable and/or alertable arrhythmia. The one or more processors are further configured to deliver, via the plurality of therapy electrodes, the therapeutic shock to the patient upon determining that the remote indication indicates that the ambulatory patient is experiencing a treatable arrhythmia. The one or more processors are further configured to record a discrepancy between the remote indication and the local indication upon determining that the remote indication indicates that the ambulatory patient is experiencing a treatable and/or alertable arrhythmia and that the local indication does not indicate that the ambulatory patient is experiencing a treatable and/or alertable arrhythmia. The one or more processors are further configured to analyze the discrepancy between the remote indication and the local indication to identify a source of the discrepancy. The one or more processors are further configured to transmit the discrepancy between the remote indication and the local indication to the remote server for identification of a source of the discrepancy.

The one or more processors are further configured to avoid delivering the therapeutic shock to the ambulatory patient upon determining that neither the remote indication nor the local indication indicates that the ambulatory patient is experiencing a treatable arrhythmia. The one or more processors are further configured to delay the therapeutic shock to the ambulatory patient upon determining that the local indication indicates that the ambulatory patient is experiencing a treatable arrhythmia but that the remote indication does not indicate that the ambulatory patient is experiencing a treatable arrhythmia. The one or more processors are further configured to perform a second analysis of the one or more ECG segments and/or of one or more additional ECG segments corresponding to the suspected arrhythmia to generate a second local indication as to whether the ambulatory patient is experiencing a treatable and/or alertable arrhythmia. The one or more ECG segments are from a first channel of the plurality of ECG electrodes, and the one or more additional ECG segments are from a second channel of the plurality of ECG electrodes. The one or more ECG segments are from a first period of time, and the one or more additional ECG segments are from a second period of time later than the first period of time. The one or more processors are further configured to transmit one or more additional ECG segments corresponding to the suspected arrhythmia to the remote server.

The one or more processors are configured to determine the one or more ECG segments corresponding to the suspected arrhythmia occurring in the ambulatory patient based on the ECG signal using a first process. The one or more processors are configured to independently analyze, at the medical device controller, the one or more ECG segments to generate a local indication as to whether the ambulatory patient is experiencing a treatable and/or alertable arrhythmia using a second process. The first process includes a lower power process than the second process. The first process includes determining whether a heart rate of the patient transgresses a predetermined threshold.

In one or more examples, a method for remotely identifying and/or verifying treatable and/or alertable cardiac arrhythmias in an ambulatory patient is performed. The method includes generating an ECG signal based on electrical signals indicative of cardiac activity in the ambulatory patient sensed via a plurality of ECG electrodes configured to be in long-term continuous contact with skin of the ambulatory patient. A wearable cardiac monitoring and treatment device configured for long-term continuous cardiac monitoring of the ambulatory patient includes the plurality of ECG electrodes. The method also includes determining one or more ECG segments corresponding to a suspected arrhythmia occurring in the ambulatory patient based on the ECG signal, transmitting the determined one or more ECG segments corresponding to the suspected arrhythmia occurring in the ambulatory patient to a remote server, and receiving, from the remote server, a remote indication based on the one or more ECG segments as to whether the ambulatory patient is experiencing a treatable and/or alertable arrhythmia. The method further includes independently analyzing, at a medical device controller of the wearable cardiac monitoring and treatment device, the one or more ECG segments to generate a local indication as to whether the ambulatory patient is experiencing a treatable and/or alertable arrhythmia, and determining whether to deliver, via a plurality of therapy electrodes of the wearable cardiac monitoring and treatment device, a therapeutic shock to the ambulatory patient based on the received remote indication and the local indication as to whether the ambulatory patient is experiencing a treatable and/or alertable arrhythmia. Implementations of the method for remotely identifying and/or verifying treatable and/or alertable cardiac arrhythmias in an ambulatory patient can include one or more of similar features and/or functionalities as the wearable cardiac monitoring and treatment system described above.

In one or more examples, a non-transitory computer-readable medium storing sequences of instructions executable by at least one processor is provided. The sequences of instructions instruct the at least one processor to remotely identify and/or verify treatable and/or alertable and/or alertable cardiac arrhythmias in an ambulatory patient. The sequences of instructions including instructions to generate an ECG signal based on electrical signals indicative of cardiac activity in the ambulatory patient sensed via a plurality of ECG electrodes configured to be in long-term continuous contact with skin of the ambulatory patient, determine one or more ECG segments corresponding to a suspected arrhythmia occurring in the ambulatory patient based on the ECG signal, and transmit the determined one or more ECG segments corresponding to the suspected arrhythmia occurring in the ambulatory patient to a remote server. The sequences of instructions also include instructions to receive, from the remote server, a remote indication based on the one or more ECG segments as to whether the ambulatory patient is experiencing a treatable and/or alertable arrhythmia, independently analyze the one or more ECG segments to generate a local indication as to whether the ambulatory patient is experiencing a treatable and/or alertable arrhythmia, and determining whether to deliver, via a plurality of therapy electrodes, a therapeutic shock to the ambulatory patient based on the received remote indication and the local indication as to whether the ambulatory patient is experiencing a treatable and/or alertable arrhythmia. Implementations of the non-transitory computer-readable medium storing sequences of instructions can include one or more of similar features and/or functionalities as the wearable cardiac monitoring and treatment system described above.

In one or more examples, a wearable cardiac monitoring and treatment system configured to remotely identify and/or verify treatable and/or alertable cardiac arrhythmias in an ambulatory patient is provided. The system includes a wearable cardiac monitoring and treatment device configured for long-term continuous cardiac monitoring of the ambulatory patient. The device includes a plurality of ECG electrodes configured to be in long-term continuous contact with skin of the ambulatory patient and sense electrical signals of the ambulatory patient indicative of cardiac activity in the ambulatory patient, a plurality of therapy electrodes configured to deliver one or more therapeutic electrical shocks to the ambulatory patient, and a medical device controller coupled to the plurality of ECG electrodes and/or the plurality of therapy electrodes. The system also includes a remote server in electronic communication with the medical device controller of the wearable cardiac monitoring and treatment device.

The medical device controller includes one or more processors configured to generate an ECG signal based on the sensed electrical signals indicative of the cardiac activity in the ambulatory patient, determine one or more ECG segments corresponding to a suspected arrhythmia occurring in the ambulatory patient based on the ECG signal, transmit the determined one or more ECG segments corresponding to the suspected arrhythmia occurring in the ambulatory patient to the remote server, independently analyze, at the medical device controller, the one or more ECG segments to generate a local indication as to whether the ambulatory patient is experiencing a treatable and/or alertable arrhythmia, upon generating a local indication that the ambulatory patient is experiencing a treatable and/or alertable arrhythmia, wait for a remote indication from the remote server as to whether the ambulatory patient is experiencing a treatable and/or alertable arrhythmia, and after waiting a predetermined amount of time and receiving no remote indication as to whether the ambulatory patient is experiencing a treatable and/or alertable arrhythmia, determine whether to deliver, via the plurality of therapy electrodes, a therapeutic shock to the ambulatory patient.

Implementations of the wearable cardiac monitoring and treatment system can include one or more of the following features. The wearable cardiac monitoring and treatment device further includes a garment configured to be worn about a torso of the ambulatory patient. The plurality of ECG electrodes and the plurality of therapy electrodes are configured to be disposed on the garment. The system further includes a gateway device, and the medical device controller is at least partially implemented in the gateway device. The one or more processors are further configured to query the remote server for the remote indication upon generating a local indication that the ambulatory patient is experiencing a treatable and/or alertable arrhythmia. The query to the remote server for the remote indication upon generating a local indication that the ambulatory patient is experiencing a treatable and/or alertable arrhythmia.

The one or more processors are configured to determine whether to deliver the therapeutic shock to the patient by performing a second independent analysis, at the medical device controller, of the one or more ECG segments and/or one or more second ECG segments to verify whether the patient is experiencing a treatable and/or alertable arrhythmia, and generating a second local indication as to whether the ambulatory patient is experiencing a treatable and/or alertable arrhythmia. The one or more ECG segments are from a first channel of the plurality of ECG electrodes, and wherein the one or more second ECG segments are from a second channel of the plurality of ECG electrodes. The one or more ECG segments are from a first period of time, and wherein the one or more second ECG segments are from a second period of time later than the first period of time. The one or more processors are further configured to initiate a treatment sequence upon generating a second local indication that the ambulatory patient is experiencing a treatable and/or alertable arrhythmia.

The one or more processors are further configured to determine whether to deliver the therapeutic shock to the patient by, upon generating a second local indication that the ambulatory patient is not experiencing a treatable and/or alertable arrhythmia, performing a third independent analysis, at the medical device controller, of the one or more ECG segments, the one or more second ECG segments, and/or one or more third ECG segments, and generating a third local indication as to whether the ambulatory patient is experiencing a treatable and/or alertable arrhythmia. The one or more ECG segments are from a first channel of the plurality of ECG electrodes, the one or more second ECG segments are from a second channel of the plurality of ECG electrodes, and the one or more third ECG segments are from a third channel of the plurality of ECG electrodes. The one or more ECG segments are from a first period of time, the one or more second ECG segments are from a second period of time later than the first period of time, and the one or more third ECG segments are from a third period of time later than the first period of time and the second period of time. The one or more processors are further configured to initiate a treatment sequence upon generating a third local indication that the patient is experiencing a treatable and/or alertable arrhythmia. The one or more processors are further configured to avoid delivering the therapeutic shock to the ambulatory patient upon generating a third local indication that the patient is not experiencing a treatable and/or alertable arrhythmia.

The one or more processors are configured to determine the one or more ECG segments corresponding to the suspected arrhythmia occurring in the ambulatory patient based on the ECG signal using a first process. The one or more processors are configured to independently analyze, at the medical device controller, the one or more ECG segments to generate a local indication as to whether the ambulatory patient is experiencing a treatable and/or alertable arrhythmia using a second process. The first process includes a lower power process than the second process. The first process includes determining whether a heart rate of the patient transgresses a predetermined threshold.

In one or more examples, a method for remotely identifying and/or verifying treatable and/or alertable cardiac arrhythmias in an ambulatory patient is performed. The method includes generating an ECG signal based on electrical signals indicative of cardiac activity in the ambulatory patient sensed via a plurality of ECG electrodes configured to be in long-term continuous contact with skin of the ambulatory patient, wherein a wearable cardiac monitoring and treatment device configured for long-term continuous cardiac monitoring of the ambulatory patient includes the plurality of ECG electrodes; determining one or more ECG segments corresponding to a suspected arrhythmia occurring in the ambulatory patient based on the ECG signal; transmitting the determined one or more ECG segments corresponding to the suspected arrhythmia occurring in the ambulatory patient to a remote server; and independently analyzing, at a medical device controller of the wearable cardiac monitoring and treatment device, the one or more ECG segments to generate a local indication as to whether the ambulatory patient is experiencing a treatable and/or alertable arrhythmia. The method also includes, upon generating a local indication that the ambulatory patient is experiencing a treatable and/or alertable arrhythmia, waiting for a remote indication from the remote server as to whether the ambulatory patient is experiencing a treatable and/or alertable arrhythmia, and after waiting a predetermined amount of time and receiving no remote indication as to whether the ambulatory patient is experiencing a treatable and/or alertable arrhythmia, determining whether to deliver, via a plurality of therapy electrodes of the wearable cardiac monitoring and treatment device, a therapeutic shock to the ambulatory patient. Implementations of the method for remotely identifying and/or verifying treatable and/or alertable and/or alertable cardiac arrhythmias in an ambulatory patient can include one or more of similar features and/or functionalities as the wearable cardiac monitoring and treatment system described above.

In one or more examples, a non-transitory computer-readable medium storing sequences of instructions executable by at least one processor is provided. The sequences of instructions instructing the at least one processor to remotely identify and/or verify treatable and/or alertable and/or alertable cardiac arrhythmias in an ambulatory patient. The sequences of instructions include instructions to generate an ECG signal based on electrical signals indicative of cardiac activity in the ambulatory patient sensed via a plurality of ECG electrodes configured to be in long-term continuous contact with skin of the ambulatory patient; determine one or more ECG segments corresponding to a suspected arrhythmia occurring in the ambulatory patient based on the ECG signal; transmit the determined one or more ECG segments corresponding to the suspected arrhythmia occurring in the ambulatory patient to a remote server; and independently analyze the one or more ECG segments to generate a local indication as to whether the ambulatory patient is experiencing a treatable and/or alertable arrhythmia. The sequences of instructions also include instructions to, upon generating a local indication that the ambulatory patient is experiencing a treatable and/or alertable arrhythmia, waiting for a remote indication from the remote server as to whether the ambulatory patient is experiencing a treatable and/or alertable arrhythmia, and after waiting a predetermined amount of time and receiving no remote indication as to whether the ambulatory patient is experiencing a treatable and/or alertable arrhythmia, determining whether to deliver, via a plurality of therapy electrodes, a therapeutic shock to the ambulatory patient. Implementations of the non-transitory computer-readable medium storing sequences of instructions can include one or more of similar features and/or functionalities as the wearable cardiac monitoring and treatment system described above.

In one or more examples, a wearable cardiac monitoring and treatment system configured to remotely identify and/or verify treatable and/or alertable cardiac arrhythmias in an ambulatory patient is provided. The system includes a wearable cardiac monitoring and treatment device configured for long-term continuous cardiac monitoring of the ambulatory patient. The device includes a plurality of ECG electrodes configured to be in long-term continuous contact with skin of the ambulatory patient and sense electrical signals indicative of cardiac activity in the patient, a plurality of therapy electrodes configured to deliver one or more therapeutic electrical shocks to the ambulatory patient, and a medical device controller coupled to the plurality of ECG electrodes and/or the plurality of therapy electrodes, and a remote server in electronic communication with the medical device controller of the wearable cardiac monitoring and treatment device.

The remote server including one or more processors is configured to receive one or more ECG segments corresponding to a suspected arrhythmia occurring in the ambulatory patient based on the sensed electrical signals indicative of the cardiac activity in the patient from the wearable cardiac monitoring and treatment device, analyze the received one or more ECG segments to determine whether the patient is experiencing a treatable and/or alertable arrhythmia, determine that the one or more ECG segments are noisy, and transmit a request to the wearable cardiac monitoring and treatment device for additional physiological data for the ambulatory patient upon determining that the one or more ECG segments are noisy.

Implementations of the wearable cardiac monitoring and treatment system can include one or more of the following features. The wearable cardiac monitoring and treatment device further includes a garment configured to be worn about a torso of the ambulatory patient. The plurality of ECG electrodes and the plurality of therapy electrodes are configured to be disposed on the garment. The system further includes a gateway device, and the medical device controller is at least partially implemented in the gateway device. The additional physiological data includes one or more additional ECG segments. The one or more ECG segments are from a first channel of the plurality of ECG electrodes, and the one or more additional ECG segments are from a second channel of the plurality of ECG electrodes. The one or more ECG segments are from a first period of time, and the one or more additional ECG segments are from a second period of time later than the first period of time. The additional physiological data includes non-ECG data. The additional physiological data includes motion data. The additional physiological data includes vibrational data.

The one or more processors are further configured to receive the additional physiological data from the wearable cardiac monitoring and treatment device and analyze the received one or more ECG segments and/or received additional physiological data to determine whether the patient is experiencing a treatable and/or alertable arrhythmia. The one or more processors are further configured to generate a remote indication as to whether the patient is experiencing a treatable and/or alertable arrhythmia and transmit the remote indication to the wearable cardiac monitoring and treatment device. The one or more processors are further configured to, upon generating a remote indication that the patient is experiencing a treatable and/or alertable arrhythmia, transmit the remote indication to a caregiver of the patient.

The one or more processors are configured to determine that the one or more ECG segments is noisy by determining that the one or more ECG segments transgress a predetermined threshold of noise. Determining that the one or more ECG segments include greater than the predetermined threshold of noise includes determining that the one or more ECG segments include a drift of at least the predetermined threshold of noise from a template baseline representation for the patient. The predetermined threshold of noise includes at least one of 10%, 15%, 20%, 25%, 30%, 35%, 40%, 45%, or 50% from the template baseline representation. The predetermined threshold of noise depends on a type of the suspected arrhythmia.

In one or more examples, a method for remotely identifying and/or verifying treatable and/or alertable and/or alertable cardiac arrhythmias in an ambulatory patient is performed. The method includes receiving, at a remote server, one or more ECG segments corresponding to a suspected arrhythmia occurring in the ambulatory patient from a wearable cardiac monitoring and treatment device. The one or more ECG segments are based on electrical signals indicative of cardiac activity of the patient sensed by a plurality of ECG electrodes configured to be in long-term continuous contact with skin of the ambulatory patient. A wearable cardiac monitoring and treatment device configured for long-term continuous cardiac monitoring of the ambulatory patient includes the plurality of ECG electrodes. The method also includes analyzing the received one or more ECG segments to determine whether the patient is experiencing a treatable and/or alertable arrhythmia, determining that the one or more ECG segments are noisy, and transmitting a request to the wearable cardiac monitoring and treatment device for additional physiological data for the ambulatory patient upon determining that the one or more ECG segments are noisy. Implementations of the method for remotely identifying and/or verifying treatable and/or alertable and/or alertable cardiac arrhythmias in an ambulatory patient can include one or more of similar features and/or functionalities as the wearable cardiac monitoring and treatment system described above.

In one or more examples, non-transitory computer-readable medium storing sequences of instructions executable by at least one processor is provided. The sequences of instructions instruct the at least one processor to remotely identify and/or verify treatable and/or alertable cardiac arrhythmias in an ambulatory patient. The sequences of instructions include instructions to receive one or more ECG segments corresponding to a suspected arrhythmia occurring in the ambulatory patient based on electrical signals indicative of cardiac activity of the patient from a wearable cardiac monitoring and treatment device. The one or more ECG segments are sensed by a plurality of ECG electrodes configured to be in long-term continuous contact with skin of the ambulatory patient. The sequences of instructions also include instructions to analyze the received one or more ECG segments to determine whether the patient is experiencing a treatable and/or alertable arrhythmia, determine that the one or more ECG segments are noisy, and transmit a request to the wearable cardiac monitoring and treatment device for additional physiological data for the ambulatory patient upon determining that the one or more ECG segments are noisy. Implementations of the non-transitory computer-readable medium storing sequences of instructions can include one or more of similar features and/or functionalities as the wearable cardiac monitoring and treatment system described above.

In one or more examples, we provide a wearable cardiac monitoring and treatment system configured to remotely identify and/or verify treatable cardiac arrhythmias in an ambulatory patient, comprising: a wearable cardiac monitoring and treatment device configured for long-term continuous cardiac monitoring of the ambulatory patient, the device comprising a plurality of ECG electrodes configured to be in long-term continuous contact with skin of the ambulatory patient and sense electrical signals indicative of cardiac activity in the ambulatory patient, a plurality of therapy electrodes configured to deliver one or more therapeutic electrical shocks to the ambulatory patient, and a medical device controller coupled to the plurality of ECG electrodes and/or the plurality of therapy electrodes, and a remote server in electronic communication with the medical device controller of the wearable cardiac monitoring and treatment device. The configuration of the wearable cardiac monitoring and treatment device and/or the medical device controller thereof in combination with the remote server may have various advantageous configurations as described herein. Likewise, corresponding methods and non-transitory computer-readable medium may also have such configurations.

### BRIEF DESCRIPTION OF THE DRAWINGS

Various aspects of at least one example are discussed below with reference to the accompanying figures, which are not intended to be drawn to scale. The figures are included to provide an illustration and a further understanding of the various aspects and examples, and are incorporated in and constitute a part of this specification but are not intended to limit the scope of the disclosure. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure.

FIG. 1 depicts an example wearable cardiac monitoring and treatment system.

FIG. 2 depicts an example of a wearable cardiac monitoring and treatment device.

FIG. 3 depicts a sample process flow for remote arrhythmia detection/verification and treatment.

FIG. 4 depicts a sample process flow for analyzing electrocardiogram (ECG) segments at a remote server to generate a remote indication as to whether a patient is experiencing a treatable and/or alertable arrhythmia.

FIG. 5 depicts a sample process flow for analyzing ECG segments at a wearable cardiac monitoring and treatment device to generate a local indication as to whether a patient is experiencing a treatable and/or alertable arrhythmia.

FIG. 6 depicts a sample process flow for determining whether to deliver a therapeutic shock to a patient wearing a wearable cardiac monitoring and treatment device based on a remote indication and a local indication.

FIG. 7 depicts another sample s process flow for remote arrhythmia detection/verification and treatment.

FIG. 8 depicts a sample process flow for determining whether to deliver a therapeutic shock to a patient after waiting a predetermined time for and not receiving a remote indication from a remote server.

FIG. 9 depicts a sample process flow for remote arrhythmia detection/verification.

FIG. 10 depicts a sample process flow for determining that one or more ECG segments are noisy.

FIG. 11A depicts an example component-level view of a medical device controller of a wearable cardiac monitoring and treatment device.

FIG. 11B depicts another example component-level view of a medical device controller of a wearable cardiac monitoring and treatment device.

FIG. 12 depicts another example of a wearable cardiac monitoring and treatment device.

FIG. 13 depicts example processing at a remote server of communications from a wearable cardiac monitoring and treatment device.

FIG. 14 depicts another example of a wearable cardiac monitoring and treatment device.

### DETAILED DESCRIPTION

Wearable medical devices, such as wearable cardiac monitoring and treatment device, are used in clinical, outpatient, or in-hospital (inpatient) care settings to monitor for treatable and/or alertable cardiac arrhythmias and provide treatment such as defibrillation, cardioversion, and/or pacing shocks in the even of life-threatening arrhythmias. In examples, clinical settings include a broad array of medical service providers and places where healthcare occurs, including urgent care centers, rehabilitation centers, nursing homes, and long-term care facilities. In examples, outpatient care settings include settings where medical procedures, tests, and/or monitoring services are provided to patients without being admitted to a hospital, e.g., such as for an overnight hospital stay. Outpatient settings can include cardiology clinics, testing centers, providers of medical procedures on an outpatient basis, wellness and prevention services at outpatient clinics, rehabilitation centers, specialized outpatient service providers (e.g., hemodialysis, chemotherapy, etc.) or other similar care providers, and/or outpatient cardiac counseling program administrators or providers. Ambulatory patients in such clinical and/or outpatient settings can be prescribed a wearable defibrillator or a wearable defibrillator or a wearable cardioverter defibrillator (WCD). In-hospital care settings, on the other hand, include settings where medical procedures, tests, and/or monitoring services are provided to a patient on admission to a hospital, e.g., for an overnight hospital stay. Such in-hospital or inpatient care settings include emergency room (ER) visits and stays, intensive care unit (ICU) stays, or settings where patients are admitted to stay for a period of time (e.g., overnight), where briefly or for an extended period of time. Patients in such in-hospital or inpatient settings can be prescribed a hospital wearable defibrillator (HWD), also described in further detail below.

A wearable cardiac monitoring and treatment device, such as a WCD or an HWD, includes therapy electrodes or defibrillator pads positioned on an upper torso of a patient. In the case of a WCD, the therapy electrodes are disposed within a garment worn about the upper torso of the patient as described in further detail below. In the case of an HWD, the therapy electrodes are disposed within pads that are adhesively attached to the upper torso of the patient. The device is configured to continuously monitor the patient's heart to detect the heart rhythm. In the event a lethal cardiac arrhythmia is detected, the device can provide the patient with predetermined alarms, e.g., a vibration and/or gong alert that indicates the patient's attention is required and that a therapeutic shock is imminent. The patient can respond to the alarms by pressing buttons or otherwise providing a response to the device to cause the device to suspend the shock. If the patient does not respond to the alarms within a certain period of time (e.g., 45 seconds to about 75 seconds), the device is configured to deliver the therapeutic shock, e.g., a defibrillation shock. The device can be configured to deliver multiple shocks in this manner so long as underlying cardiac signals indicate an ongoing arrhythmia condition in the patient.

In such wearable cardiac monitoring and treatment devices, the device must be able to monitor the patient's heart rhythm (e.g., the patient's ECG data) and detect, from the patient's heart rhythm, when the patient is experiencing a treatable and/or alertable arrhythmia. For example, treatable arrhythmias can include those arrhythmias for which the cardiac device is configured to issue one or more therapeutic shocks in order to restore normal sinus rhythm. For example, treatable arrhythmias can include VT or VF treatable by cardioversion and/or defibrillation shocks, or bradycardia, tachycardia, or asystole treatable by one or more transcutaneous pacing therapy routines. In some examples, a cardiac monitor may provide an alert that an arrhythmia is occurring in the patient. For example, such alertable arrhythmias can include an onset of a bradycardia, atrial fibrillation, tachycardia, and pauses, among others. Such devices may also be in communication with a remote server and transmit data, such as ECG data, to the remote server. For example, such communication with a remote server involves transmission and/or reception of detailed and/or summary ECG data, ECG event flags, associated time stamps, patient event history information, event flags, patient self-reported event ECG data and associated timestamps (e.g., when a patient self-reports a pause, racing heart, fatigue, chest pain, light-headedness, or other symptom), and device service reminders and device event flags. In implementations, such communications include long range communications technologies (e.g., cellular communications) and/or a mix of short range and long range communications technologies (e.g., Bluetooth® protocol-based communications, Wi-Fi based communication, or combinations thereof) as described in further detail below.

As such, in implementations herein, the remote server may also play a role in detecting when the patient is experiencing a treatable and/or alertable arrhythmia. However, issues may arise, for example, in the communication of ECG data between the device and the remote server that the remote server needs to help the device detect treatable and/or alertable cardiac arrhythmias (e.g., communication networks may be unreliable, e.g., experience dropped data packets, causing data to be unsent between the wearable cardiac monitoring and treatment device and the remote server). Further, ECG analysis algorithms disposed within a remote server processor and a device processor may disagree on whether the ECG data shows that the patient is experiencing a treatable and/or alertable arrhythmia, or the ECG data may be too noisy for the remote server processor to use to determine whether the patient is experiencing a treatable and/or alertable arrhythmia.

As such, this disclosure relates to a wearable cardiac monitoring and treatment system configured to remotely identify and/or verify treatable and/or alertable cardiac arrhythmias in an ambulatory patient. In implementations, a patient is prescribed a wearable cardiac monitoring and treatment device configured for long-term continuous cardiac monitoring of the ambulatory patient. The cardiac device includes ECG electrodes configured to be in long-term continuous contact with skin of the ambulatory patient and sense electrical signals indicative of cardiac activity in the ambulatory patient. The cardiac device also includes therapy electrodes configured to deliver one or more therapeutic electrical shocks to the ambulatory patient, as well as a medical device controller coupled to the plurality of ECG electrodes and/or the plurality of therapy electrodes. The medical device controller includes one or more processors in communication with a memory disposed, for example, in the medical device controller. The medical device controller is further in communication a remote server via a network.

In implementations, the medical device controller is configured to generate at least one ECG signal (e.g., in a single ECG channel device) based on the sensed electrical signals indicative of cardiac activity in the patient from the ECG electrodes. In examples, the controller can generate multiple ECG signals, e.g., 2 ECG channels or more based on 2 or more ECG electrodes, 3 ECG channels or more based on 3 or more ECG electrodes, or 4 ECG channels or more based on 4 or more ECG electrodes disposed about the patient's body. Using the at least one ECG signal, the medical device controller determines one or more segments that correspond to a suspected arrhythmia in the patient. For example, such a segment may be about 1 minute (60 seconds), 90 seconds, 2 minutes (120 seconds), 3 minutes, 5 minutes, or other user-provided duration. For example, the duration of such a segment can be based on the duration of the underlying suspected arrhythmia event. Additionally or alternatively, the duration can include a certain pre-event period of time and a certain post-event period of time. Such pre-event and/or post-event periods of time can also be 1 minute (60 seconds), 90 seconds, 2 minutes (120 seconds), 3 minutes, 5 minutes, or other user-provided duration. In an example scenario, if the suspected arrhythmia event initiated at time t, and is ongoing in the patient, the controller can determine that the corresponding ECG segment begins at around time t-30 seconds and includes around 120 seconds of the arrhythmia for a total ECG segment duration of around 150 seconds.

The medical device controller transmits these one or more ECG segments to the remote server and continues processing these one or more ECG segments in analyzing the one or more ECG segments for confirmation of the suspected arrhythmia. More specifically, the medical device controller independently analyzes the one or more ECG segments to generate a local indication as to whether the patient is experiencing a treatable and/or alertable arrhythmia, e.g., confirms the suspected arrhythmia as a treatable and/or alertable arrhythmia in the patient. The medical device controller also waits for a similar remote indication from the remote server as to whether the patient is experiencing a treatable and/or alertable arrhythmia. For example, the remote server performs its own, independent analysis of the one or more ECG segments to determine whether the patient is experiencing a treatable and/or alertable arrhythmia. In examples, the remote server analysis can be based on a same or similar arrhythmia detection process than that performed by the medical device controller. In examples, the remote server analysis can be based on a different arrhythmia detection process than that performed by the medical device controller. For example, the different arrhythmia detection process executed on the remote server can be based on a more sensitive (e.g., higher sensitivity in output with lower rate of false negative results, such as 99% sensitivity on server versus 95% sensitivity on controller), a more specific (e.g., higher specificity in output with lower rate of false positive results, such as 99% specificity on server versus 95% specificity on controller), and/or use different and/or additional physiological parameters, and/or different methodology (e.g., machine learning classifier based approach) than the arrhythmia detection process executed by the medical device controller. Based on the local indication generated by the medical device controller and the remote indication generated by the remote server as to whether the patient is experiencing a treatable and/or alertable arrhythmia, the medical device controller determines whether to deliver a therapeutic shock to the patient.

For example, if the local indication and the remote indication agree that the patient is not experiencing a treatable and/or alertable arrhythmia, the medical device controller may not perform any further actions and return to monitoring the patient's cardiac rhythm. As another example, if the local indication and the remote indication agree that the patient is experiencing a treatable and/or alertable arrhythmia, the medical device controller may initiate a treatment sequence (e.g., by alerting the patient that a defibrillation shock is imminent, as described above, and preparing to deliver the defibrillation shock via the therapy electrodes). As another example, if the local indication and the remote indication do not agree as to whether the patient is experiencing a treatable and/or alertable arrhythmia, the medical device controller may perform further analysis of the patient's cardiac and/or non-cardiac physiological data to confirm whether the patient is experiencing a treatable and/or alertable arrhythmia. Alternatively, or additionally, the medical device controller may transmit additional cardiac and/or non-cardiac physiological data to the remote server such that the remote server can confirm whether the patient is experiencing a treatable and/or alertable arrhythmia. In implementations, the medical device controller and/or the remote server may also record the discrepancy between the remote and local indications and analyze the discrepancy to determine if, for example, an update can be made to instructions used by the medical device controller to help ensure agreement between the medical device controller and the remote server in the future.

In implementations, the medical device controller is configured to generate an ECG signal, determine one or more ECG segments corresponding to a suspected arrhythmia, and transmit the determined one or more ECG segments to the remote server. The medical device controller also independently analyzes the one or more ECG segments to generate a local indication as to whether the patient is experiencing a treatable and/or alertable arrhythmia. The medical device controller then waits for the remote indication from the remote server as to whether the patient is experiencing the treatable and/or alertable arrhythmia. After waiting a predetermined amount of time and receiving no remote indication, the medical device controller is configured to determine whether to deliver a therapeutic shock to the patient. For instance, the medical device controller may simply act on its own determination of whether the patient is experiencing a treatable and/or alertable arrhythmia (e.g., initiating a treatment sequence if the local indication indicates that that patient is experiencing a treatable arrhythmia, or providing an alert that the arrhythmia is occurring if the device is configured to provide an alert about the arrhythmia). As another example, the medical device controller may run one or more follow-up analyses to confirm whether the patient is experiencing a treatable and/or alertable arrhythmia. If the medical device controller receives the remote indication during these analyses, the medical device controller may revert to determining whether the remote indication and local indication agree as to whether the patient is experiencing a treatable and/or alertable arrhythmia, as described above.

In implementations, the remote server receives the one or more ECG segments from the cardiac device and analyzes the one or more ECG segments to determine whether the patient is experiencing a treatable and/or alertable arrhythmia. However, while analyzing the one or more ECG segments, the remote server may determine that the ECG segments are too noisy for the remote server to confirm whether the patient is experiencing a treatable and/or alertable arrhythmia (e.g., within a predetermined confidence level). As such, the remote server may request additional data from the cardiac device to resolve the issue of the noisy ECG segments. The additional data may include one or more additional ECG segments (e.g., ECG segments from another channel of the ECG electrodes and/or more current ECG segments) and/or non-ECG data, such as motion data, vibrational data, posture data, respiration data, temperature data, humidity data, and/or the like.

In one example use case, a clinician or other caregiver prescribes that a patient at risk of heart failure wear a wearable cardiac monitoring and treatment device for a certain amount of time (e.g., until the patient is scheduled for surgery to receive an implantable cardiac defibrillator). The cardiac device continuously monitors the patient for treatable cardiac arrhythmias, such as ventricular fibrillation. As the patient is wearing the cardiac device, the cardiac device determines a section of ECG data that the cardiac device suspects, based on a first cardiac arrhythmia analysis, shows the patient is experiencing a treatable cardiac arrhythmia. The cardiac device transmits the section of ECG data to the remote server while it performs a second cardiac arrhythmia analysis (e.g., a more computationally intensive cardiac arrhythmia analysis) on the section of ECG data to confirm whether the patient is experiencing the treatable arrhythmia. The cardiac device outputs a local indication that the patient is not experiencing a treatable arrhythmia. However, the cardiac device receives a remote indication from the remote server indicating that the patient is experiencing a treatable arrhythmia. While a predetermined amount of time from the cardiac device's first determination suspecting the patient of experiencing a cardiac arrhythmia has not expired (e.g., after which the cardiac device will automatically initiate a treatment sequence), the cardiac device transmits a more current segment of ECG data to the remote server while the cardiac device analyzes the same to determine if the patient is experiencing a cardiac arrhythmia. The cardiac device now outputs a local indication that the patient is experiencing the cardiac arrhythmia and receives a remote indication from the remote server in agreement. As such, the cardiac device initiates a treatment sequence for the patient, beginning by activating one or more haptic, verbal, and/or visual alarms warning the patient that the cardiac device will imminently provide a defibrillation shock to the patient.

In another example use case, a clinician or other caregiver prescribes that a patient at risk of heart failure wear a wearable cardiac monitoring and treatment device for an extended period of time because surgery to receive an implantable cardiac defibrillator is too risky for the patient. While the patient is wearing the cardiac device, the cardiac device determines from the patient's ECG data that the patient is experiencing an episode of bradycardia. The cardiac device outputs a local indication to the same, segments the ECG data corresponding to the detected cardiac arrhythmia, and transmits the ECG segment to the remote server. The cardiac device then waits for a predetermined amount of time from the detection of the episode of bradycardia for the remote server for the remote indication (e.g., 5 minutes). The predetermined amount of time passes without receive the remote indication. The cardiac device then performs another analysis on ECG data from another ECG channel of the ECG electrodes to verify whether the patient is experiencing the episode of bradycardia. On confirming that the patient is experiencing an episode of bradycardia, and still having not received the remote indication, the cardiac device proceeds with activating a treatment sequence for the patient. For example, the cardiac device delivers antibradycardiac pacing pulses to the patient via the therapy electrodes.

In another example use case, a clinician or other caregiver prescribes that a patient at risk of heart failure wear a wearable cardiac monitoring and treatment device while the patient is staying in a hospital. While the patient is wearing the cardiac device, the cardiac device determines from the patient's ECG data that the patient is experiencing ventricular fibrillation. The cardiac device transmits an ECG segment corresponding to the suspected ventricular fibrillation to the remote server. The remote server analyzes the ECG segment according to a machine learning process to determine whether the patient is experiencing ventricular fibrillation. However, the remote server categorizes the ECG segment as noise, possibly from patient movement. The remote server then requests motion data (e.g., from an accelerometer) and/or a more current ECG segment from the cardiac device to confirm this analysis as noise. The remote server receives the motion data and/or the more current ECG segment. After performing the machine learning process on the more current ECG segment, the remote server again categorizes the segment as noise. The remote server also analyzes the patient's motion data by determining accelerometer counts for the patient and the patient's posture, which show that the patient is upright and exhibiting accelerometer counts consistent with walking. As such, the remote server determines that the noise is due to patient movement and outputs a remote indication that the patient is not experiencing a treatable arrhythmia, which the remote server transmits back to the cardiac device.

The wearable cardiac monitoring and treatment system, devices, techniques, and methods described herein provide several advantages. In one scenario, implementations described herein can minimize false alarms. For example, by performing an analysis of whether the patient is experiencing a treatable and/or alertable arrhythmia on the cardiac device and on the remote server in communication with the cardiac device, the system may minimize false alarms, e.g., by better identifying treatable and/or alertable arrhythmias. In another example, implementations described herein can allow for development of computationally intensive arrhythmia detection algorithms that are deployed on the server rather than the controller. Such algorithms may need components that are appreciable larger sized, such as increased power capacity. The system may be able to use more computationally intensive arrhythmia detection processes than could be implemented on the cardiac device without, for example, increasing the bulk of the cardiac device or decreasing the battery life. For example, the system may be able to detect more types of arrhythmias, such as atrial fibrillation and bradycardia, in addition to ventricular fibrillation and implement treatment plans for these additional types of arrhythmias. At the same time, by having a robust process for analyzing cardiac data on the cardiac device, the system may avoid risks to the patient's health that could arise if the cardiac device suspects that the patient is experiencing a treatable and/or alertable arrhythmia but is unable to establish a communication link between the remote server.

In implementations, some patients may be more at risk of experiencing an arrhythmia than others or certain types of arrhythmias than others. As such, the cardiac device may include the capability to turn the remote arrhythmia detection capabilities on or off. For patients less at risk, a caregiver may choose to keep the remote arrhythmia detection capabilities turned off. However, for patients more at risk of arrhythmias in general, or more at risk of a certain type of arrhythmia, the caregiver may keep the remote arrhythmia detection capabilities turned on. The remote server may thus help confirm any arrhythmias the patient may be experiencing, which may help decrease false alarms and/or allow for more accurate detection of the specific type of arrhythmia the patient is more likely to be experiencing.

In implementations, more comprehensive arrhythmia analysis capabilities may be moved to the remote server, while the local cardiac device retains more limited arrhythmia analysis capabilities. For example, the local cardiac device may retain the capability of detecting only ventricular fibrillation or ventricular tachycardia events, while the remote server may contain the ability to detect a wider array of arrhythmias and ECG patterns, such as premature ventricular contractions (PVCs), atrial fibrillation, bradycardia, pauses, asystole, among others. This may allow the local cardiac device to be downsized (e.g., due to fewer memory and battery requirements), which may result in a more comfortable and more portable device for patients to continuously wear.

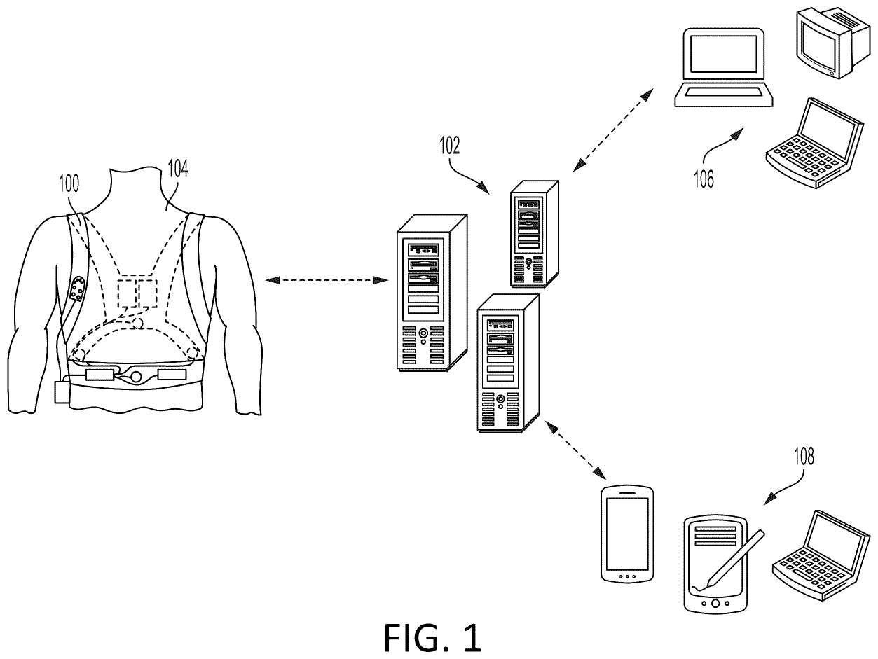

FIG. 1 shows a wearable cardiac monitoring and treatment system that includes a wearable cardiac monitoring and treatment device 100 in communication with a remote server 102. The cardiac device 100 is configured for long-term continuous cardiac monitoring of an ambulatory patient 104. In implementations, the cardiac device 100 may be implemented as a wearable garment configured to be worn continuously by the patient 104 for an extended period of time. The cardiac device 100 may include one or more electrocardiogram (ECG) sensing electrodes configured to be in long-term continuous contact with skin of the patient 104 and sense electrical signals indicative of cardiac activity in the patient 104. In implementations, the cardiac device 100 may include one or more other externally worn sensors configured to be disposed on the garment and output one or more physiological signals for the patient 104 and/or for the environment of the patient 104, such as biovibrational sensors, photoplythysmography sensors, radiofrequency (RF) sensors (which may be used, for example, to determine lung fluid content in the patient 104), temperature sensors, humidity sensors, and/or the like. The cardiac device 100 may further include one or more therapy electrodes configured to deliver one or more therapeutic electrical shocks to the patient 104.

The cardiac device 100 is configured to transmit signals and data generated by the cardiac device 100 to the remote server 102. Accordingly, the cardiac device 100 may be in wireless communication with the remote server 102. As an illustration, the cardiac device 100 may communicate with the remote server 102 via cellular networks, via Bluetooth®-to-TCP/IP access point communication, via Wi-Fi, and the like. As such, the cardiac device 100 may include communications circuitry configured to implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the communications circuitry in the cardiac device 100 may be part of an Internet of Things (IoT) and communicate with the remote server 102 via IoT protocols (e.g., Constrained Application Protocol (CoAP), Message Queuing Telemetry Transport (MQTT), Wi-Fi, Zigbee, Bluetooth®, Extensible Messaging and Presence Protocol (XMPP), Data-Distribution Service (DDS), Advanced Messaging Queuing Protocol (AMQP), and/or Lightweight M2M (LwM2M)).

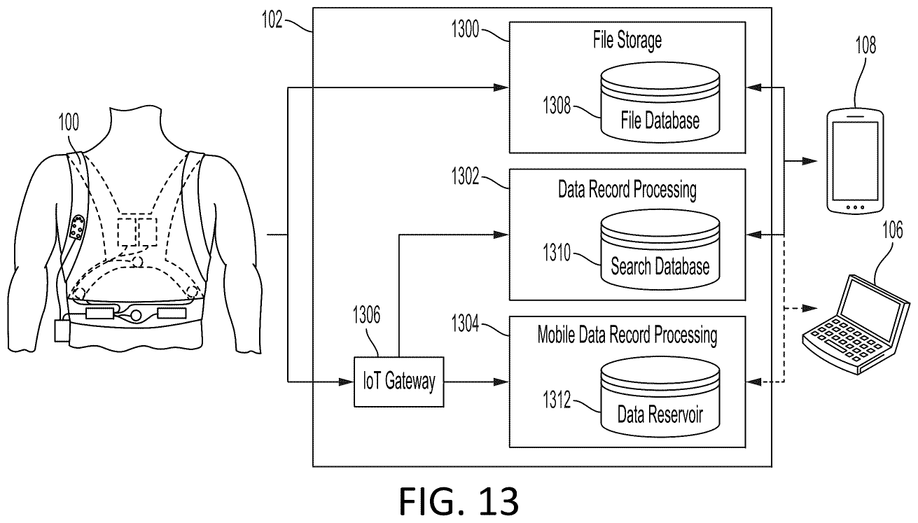

The remote server 102 is configured to receive and process signals and data transmitted by the cardiac device 100 worn by the patient 104. Accordingly, the remote server 102 may include a computing device, or a network of computing devices, including at least one database (e.g., implemented in non-transitory media or memory) and at least one processor configured to execute sequences of instructions (e.g., stored in the database, with the at least one processor being in communication with the database) to receive and process the signals transmitted by the cardiac device 100. The database may be implemented as flash memory, solid state memory, magnetic memory, optical memory, cache memory, combinations thereof, and/or others. The remote server 102 also includes at least one processor. Referring briefly to FIG. 13, the remote server 102 may include, for example, file storage 1300, data record processing 1302, mobile data record processing 1304, and an IoT gateway 1306, as described in further detail below. The at least one processor of the remote server 102 can be implemented via the file storage 1300, data record processing 1302, mobile data record processing 1304, and/or IoT gateway 1306. In implementations, the at least one processor of the remote server 102 can be a digital signal processor (DSP) such as a 24-bit DSP processor. As another example, the at least one processor of the remote server 102 can be a multi-core processor, e.g., having two or more processing cores. As another example, the at least one processor of the remote server 102 can be an Advanced RISC Machine (ARM) processor, such as a 32-bit ARM processor. The at least one processor of the remote server 102 can execute an embedded operating system and further execute services provided by the operating system, where these services can be used for file system manipulation, display and audio generation, basic networking, firewalling, data encryption, communications, and/or the like.

Returning to FIG. 1, the wearable cardiac monitoring and treatment system further includes one or more user interfaces, such as one or more technician interfaces 106 and one more caregiver interfaces 108. The technician interfaces 106 and caregiver interfaces 108 are in electronic communication with the remote server 102 via a wired or wireless connection. For instance, the technician interfaces 106 and caregiver interfaces 108 may communicate with the remote server 102 via Wi-Fi, via Ethernet, via cellular networks, and/or the like. The technician interfaces 106 and caregiver interfaces 108 may include, for example, desktop computers, laptop computers, and/or portable personal digital assistants (e.g., smartphones, tablet computers, etc.).

The technician interfaces 106 and the caregiver interfaces 108 are configured to electrically communicate with the remote server 102, for example, for the purpose of reviewing arrhythmia information for the patients wearing a cardiac device 100. Accordingly, each of a technician interface 106 and a caregiver interface 108 may include a computing device having a processor connected to a memory and a visual display. In implementations, the technician interface 106 may display to a user of the technician interface 106 (e.g., a technician) arrhythmia data for the patient 104. Similarly, in implementations, the caregiver interface 108 may display to a user of the caregiver interface 108 (e.g., a doctor, a clinician, a relative caregiver, etc.) notifications about detected arrhythmias that the patient 104 is experiencing and/or has experienced. In implementations, as shown in FIG. 13, the technician interfaces 106 and the caregiver interfaces 108 may be configured to have different levels of access to the remote server 102. For example, the technician interfaces 106 may be able to access information stored in the file storage 1300, the data record processing 1302, and the mobile data record processing 1304, but the technician interfaces 106 may only have access to information stored in the file storage 1300 and the data record processing 1302.

In some implementations, a technician interface 106 and/or a caregiver interface 108 may be a specialized user interface configured to communicate with the remote server 102. As an example, the technician interface 106 may be a specialized computing device configured to communicate with the remote server 102 to send and receive arrhythmia information for patients wearing a cardiac devices 100. As another example, the caregiver interface 108 may be a user interface specialized to receive alerts or notifications related to detected arrhythmias in patients wearing a cardiac device 100. In such implementations, the specialized technician interface 106 and/or caregiver interface 108 may be limited to performing functions related to the wearable cardiac monitoring and treatment system.

In some implementations, a technician interface 106 and/or a caregiver interface 108 may be a generalized user interface that has been adapted to communicate with the remote server 102. To illustrate, the technician interface 106 may be a computing device (e.g., a laptop, a portable personal digital assistant such as a smartphone or tablet, etc.) executing a technician application that configures the computing device to communicate with the remote server 102. For example, the technician application may be downloaded from an application store or otherwise installed on the computing device. Accordingly, when the computing device executes the technician application, the computing device is configured to establish an electronic communication link with the remote server 102 to receive and transmit arrhythmia information for patients wearing a cardiac device 100. Similarly, the caregiver interface 108 may be a computing device (e.g., a laptop, a portable personal digital assistant such as a smartphone or tablet, etc.) executing a caregiver application that configures the computing device to communicate with the remote server 102. The caregiver application may be likewise downloaded from an application store or otherwise installed on the computing device and, when executed, may configure the computing device to establish a communication link with the remote server 102 to receive and display arrhythmia information and/or notifications or alerts regarding detected arrhythmias for patients wearing a cardiac device 100.

The application store is typically included within an operating system of a computing device implementing a user interface. For example, in a device implementing an operating system provided by Apple Inc. (Cupertino, California), the application store can be the App Store, a digital distribution platform, developed and maintained by Apple Inc., for mobile apps on its iOS and iPadOS® operating systems. The application store allows a user to browse and download an application, such as the technician or caregiver application, developed in accordance with the Apple® iOS Software Development Kit. For instance, such technician or caregiver application may be downloaded on an iPhone® smartphone, an ipod Touch® handheld computer, or an iPad® tablet computer, or transferred to an Apple Watch® smartwatch. Other application stores may alternatively be used for other types of computing devices, such as computing devices operating on the Android® operating system.

In some implementations, the technician application and the caregiver application may be the same application, and the application may provide different functionalities to the computing device executing the application based on, for example, credentials provided by the user. For instance, the application may provide technician functionalities to a first computing device in response to authenticating technician credentials entered on the first computing device, and may provide caregiver functionalities to a second computing device in response to authenticating caregiver credentials entered on the second computing device. In other case, the technician application and the caregiver application may be separate applications, each providing separate functionalities to a computing device executing them.

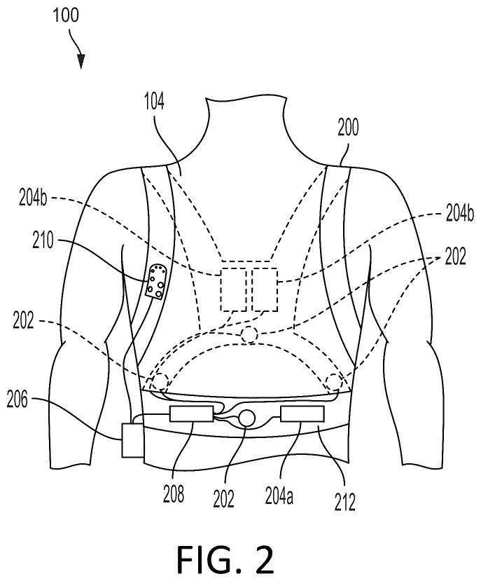

FIG. 2 shows the wearable cardiac monitoring and treatment device 100, according to various implementations. As shown in FIG. 2, the cardiac device 100 is external and wearable by the patient 104 around the patient's torso. Such a cardiac device 100 can be, for example, capable and designed for moving with the patient 104 as the patient 104 goes about their daily routine. For example, the cardiac device 100 may be configured to be bodily-attached to the patient 104. As noted above, the cardiac device 100 may be a wearable defibrillator or a wearable cardioverter defibrillator. In one example scenario, such wearable defibrillators can be worn nearly continuously or substantially continuously for a week, two weeks, a month, or two or three months at a time. During the period of time in which they are worn by the patient 104, the wearable defibrillators can be configured to continuously or substantially continuously monitor the vital signs of the patient 104 and can be configured to, upon determination that treatment is required, deliver one or more therapeutic electrical pulses to the patient 104. For example, such therapeutic shocks can be pacing, defibrillation, cardioversion, or transcutaneous electrical nerve stimulation (TENS) pulses.

As shown in FIG. 2, the wearable cardiac monitoring and treatment device 100 can include one or more of the following: a garment 200 configured to be worn about the patient's torso, one or more ECG electrodes 202 configured to be disposed on the garment 200 and further configured to sense ECG signals indicative of cardiac activity in the patient 104, one or more therapy electrodes 204a and 204b (collectively referred to herein as therapy electrodes 204) configured to be disposed on the garment 200, a medical device controller 206, a connection pod 208, a patient interface pod 210, a belt 212, or any combination of these. In implementations, the wearable cardiac monitoring and treatment device 100 may also include additional sensors, such as one or more motion detectors configured to generate motion data indicative of physical activity performed by the patient 104, one or more wear state sensors configured to detect a wear state of the wearable cardiac monitoring and treatment device 100, one or more bioacoustics sensors configured to generate bioacoustics signals for the heart of the patient 104, one or more respiration sensors configured to generate respiration signals indicative of the respiration activity of the patient 104, and/or the like.

In examples, at least some of the components of the cardiac device 100 can be configured to be disposed on the garment 200 by being removably mounted on or affixed to the garment 200, such as by mating hooks, hook-and-loop fabric strips, receptacles (e.g., pockets), snaps (e.g., plastic or metal snaps), and/or the like. For instance, the ECG electrodes 202 may be removably attached to the garment 200 by hook-and-loop fabric strips on the ECG electrodes 202 and on the garment 200, and the therapy electrodes 204 may be removably attached on the garment 200 by being inserted into receptacles of the garment 200. In some examples, at least some of the components of the cardiac device 100 can be permanently integrated into the garment 200, such as by being sewn into the garment or by being adhesively secured to the garment 200 with a permanent adhesive. In examples, at least some of the components may be connected to each other through cables, through sewn-in connections (e.g., wires woven into the fabric of the garment 200), through conductive fabric of the garment 200, and/or the like.

The medical device controller 206 can be operatively coupled to the ECG electrodes 202 and the therapy electrodes 204, which can be temporarily and/or removably affixed to the garment 200 (e.g., assembled into the garment 200 or removably attached to the garment 200, for example, using hook-and-loop fasteners) and/or permanently integrated into the garment 200, as discussed above. As shown in FIG. 2, the ECG electrodes 202 and/or the therapy electrodes 204 can be directly operatively coupled to the medical device controller 206 and/or operatively coupled to the medical device controller 206 through the connection pod 208. Component configurations other than those shown in FIG. 2 are also possible. For example, the ECG electrodes 202 can be configured to be attached at various positions about the body of the patient 104. In some implementations, the ECG electrodes 202 and/or at least one of the therapy electrodes 204 can be included on a single integrated patch and adhesively applied to the patient's body. In some implementations, the ECG electrodes 202 and/or at least one of the therapy electrodes 204 can be included in multiple patches and adhesively applied to the patient's body. Such patches may be in a wired (e.g., via the connection pod 208) or wireless connection with the medical device controller 206.

As discussed above, the ECG electrodes 202 can be configured to detect electrical signals indicative of cardiac activity of the patient 104. Example ECG electrodes 202 may include a metal electrode with an oxide coating such as tantalum pentoxide electrodes. For example, by design, the ECG electrodes 202 can include skin-contacting electrode surfaces that may be deemed polarizable or non-polarizable depending on a variety of factors including the metals and/or coatings used in constructing the electrode surface. All such electrodes can be used with the principles, techniques, devices and systems described herein. For example, the electrode surfaces can be based on stainless steel, noble metals such as platinum, or Ag—AgCl.

In implementations, the ECG electrodes 202 can be used with an electrolytic gel dispersed between the electrode surface and the patient's skin. In implementations, the ECG electrodes 202 can be dry electrodes that do not need an electrolytic material. As an example, such a dry electrode can be based on tantalum metal and having a tantalum pentoxide coating, as is described above. Such dry electrodes can be more comfortable for long-term monitoring applications.

In implementations, the ECG electrodes 202 can include additional components such as accelerometers, acoustic signal detecting devices, and/or other measuring devices for recording additional parameters. For example, the ECG electrodes 202 can also be configured to detect other types of patient physiological parameters and acoustic signals, such as tissue fluid levels, heart vibrations, lung vibrations, respiration vibrations, patient movement, etc. In implementations, the cardiac device 100 may include sensors or detectors separate from the ECG electrodes 202, such as separate motion detector(s), wear state detector(s), bioacoustics sensor(s), respiration sensor(s), temperature sensor(s), pressure sensor(s), and/or the like. In some examples, the therapy electrodes 204 can also be configured to include sensors configured to detect ECG signals as well as, or in the alternative, other physiological signals from the patient 104.

The connection pod 208 can, in some examples, include a signal processor configured to amplify, filter, and/or digitize cardiac signals, such as the ECG signals, prior to transmitting the cardiac signals to the medical device controller 206. One or more therapy electrodes 204 can be configured to deliver one therapeutic electrical more (e.g., cardioversion/defibrillation) shocks to the body of the patient 104 when the cardiac device 100 determines that such treatment is warranted based on the signals detected by the ECG electrodes 202 and processed by the medical device controller 206. Example therapy electrodes 204 can include conductive metal electrodes such as stainless-steel electrodes that include, in certain implementations, one or more conductive gel deployment devices configured to deliver conductive gel between the metal electrode and the patient's skin prior to delivery of a therapeutic shock.

In implementations, the medical device controller 206 may also be configured to warn the patient 104 prior to the delivery of a therapeutic shock, such as via output devices integrated into or connected to the medical device controller 206, the connection pod 208, and/or the patient interface pod 210. The warning may be auditory (e.g., a siren alarm, a voice instruction indicating that the patient 104 is going to be shocked), visual (e.g., flashing lights on the medical device controller 206), haptic (e.g., a tactile, buzzing alarm generated by the connection pod 208), and/or the like. If the patient 104 is still conscious, the patient 104 may be able to delay or stop the delivery of the therapeutic shock. For example, the patient 104 may press one or more buttons on the patient interface pod 210 to indicate that the patient 104 is still conscious. In response to the patient 104 pushing the one or more buttons, the medical device controller 206 may delay or stop the delivery of the therapeutic shock.

In implementations, the cardiac device 100 may also be configured to communicate with a gateway device, where the gateway device is configured to facilitate communication between the cardiac device 100 and the remote server 102. To illustrate, the gateway device may be a portable personal digital assistant (e.g., a smartphone, a tablet computer, etc.) configured to communicate between the cardiac device 100 and the remote server 102. For example, the gateway device may receive data from the cardiac device 100 via a local network (e.g., via Bluetooth®, etc.), such as ECG segments, and transmit the data to the remote server 102 via a wide area network (e.g., via Wi-Fi, via cellular communication networks, etc.). In embodiments, the gateway device may do at least some of the analysis and processing described above and below such that the medical device controller 206 is at least partially implemented in the gateway device. As an illustration, the gateway device may analyze the patient's ECG signal to determine whether the patient 104 is suspected of experiencing an arrhythmia.

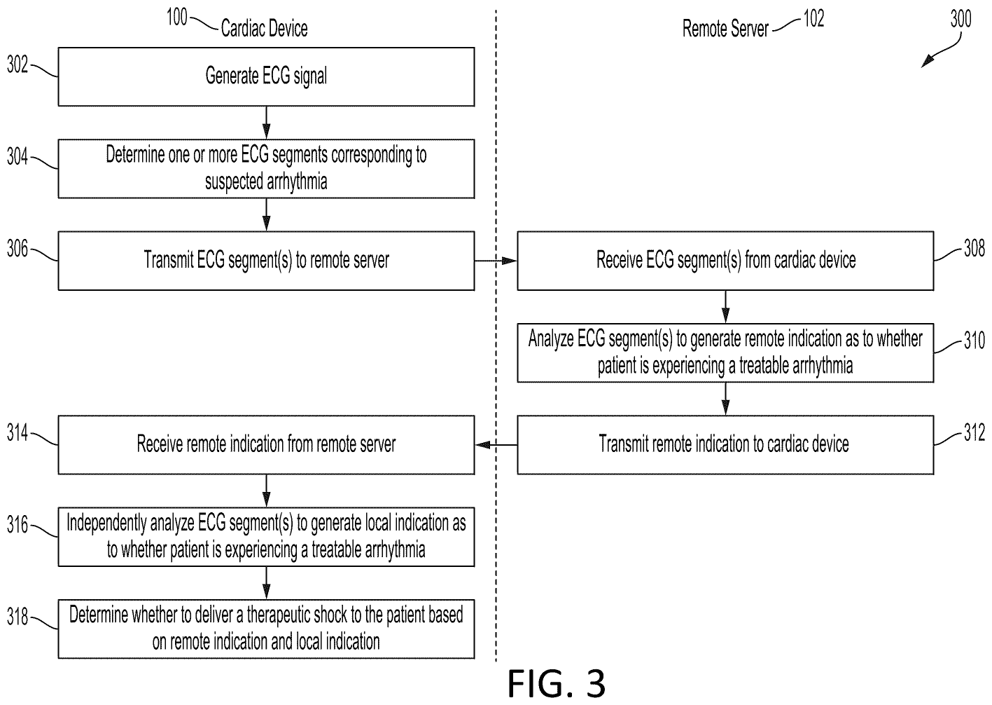

FIG. 3 illustrates a sample process flow for remote arrhythmia detection/verification and treatment. The sample process 300 can be implemented by the cardiac device 100 (e.g., the medical device controller 206) and by the remote server 102 in communication with the cardiac device 100 (e.g., at least one processor of the remote server 102), as shown in FIG. 3. The cardiac device 100 generates an ECG signal at step 302. In implementations, as described above, the ECG electrodes 202 of the cardiac device 100 sense electrical signals indicative of cardiac activity in the patient 104. The cardiac device 100 then generates an ECG signal based on the sensed electrical signals from the ECG electrodes 202. In implementations, the cardiac device 100 may generate more than one ECG signal. For instance, the ECG electrodes 202 may form multiple ECG channels (e.g., from combinations of different pairs of the ECG electrodes 202), and the cardiac device 100 may generate an ECG signal for each ECG channel.

The cardiac device 100 determines one or more ECG segments corresponding to a suspected arrhythmia in the patient 104 at step 304. In implementations, the cardiac device 100 continuously analyzes the ECG signal to identify whether a portion of the ECG signal is consistent with one or more signs that the patient 104 is experiencing a cardiac arrhythmia. For example, the cardiac device 100 may use a QRS detector on the ECG signal to assess the patient's heart rate. If the patient's heart rate transgresses a predetermined threshold (e.g., at or above 150, 160, 170, etc. bpm for ventricular fibrillation or ventricular tachycardia; at or below 30, 20, 10, etc. bpm for bradycardia), the cardiac device 100 may suspect that the patient 104 is having an arrhythmia. In implementations, the predetermine threshold may be user-configurable. As another example, the cardiac device 100 may additionally use motion data from a motion sensor of the cardiac device 100 to determine if the patient 104 is currently engaging in physical activity. The cardiac device 100 may then suspect that the patient 104 is having an arrhythmia if the patient's heart rate is above the predetermined threshold and the patient is not engaging in physical activity. Once the cardiac device 100 determines that the patient 104 is suspected of having an arrhythmia, the cardiac device 100 may segment the ECG signal corresponding to the suspected arrhythmia. For instance, the cardiac device 100 may create one or more segments of predetermined length (e.g., 30 seconds) starting a certain amount of time before the suspected arrhythmia and continuing into the arrhythmia.

The cardiac device 100 transmits the one or more ECG segments corresponding to the suspected arrhythmia occurring in the patient 104 to the remote server 102 at step 306. For instance, the cardiac device 100 may transmit a data structure including the ECG data corresponding to the suspected arrhythmia, as well as additional data relating to the patient 104, the ECG electrodes 202, interpretation of the ECG data, and/or the like. An example of a data structure that may be used for storing and transmitting an ECG segment is in Table 1 provided below:

TABLE 1
Example ECG Segment Data Structure

| Field | | | |
|---|---|---|---|
| Number | Required? | Field Name | Description |
| | Yes | Checksum | 2 bytes - Checksum over entire record |
| | Yes | Size | 4 bytes - (Unsigned) size of entire ECG |
| | | | record (in bytes) |
| Section 0 | Yes | Pointers | Pointers to data areas in the record |
| Section 1 | Yes | Header | Header information for patient data/ECG |
| | | Information | data |
| Section 2 | Dependent | Encoding Tables | Data from encoding the ECG data and/or |
| | | | reference data (if used) |
| Section 3 | Yes | ECG Lead | Identifies which ECG leads are contained |
| | | Definition | within the record |
| Section 4 | No | QRS Locations | If reference data is encoded, shows position |
| | | | of reference data relative to encoded rhythm |
| | | | data from Section 6 |
| Section 5 | No | Reference Beat | Encoded reference data |
| | | Data | |
| Section 6 | Yes | Rhythm Data | Encoded ECG data |
| Section 7 | No | Global | Global measurements |
| | | Measurements | |
| Section 8 | No | Textual Diagnosis | Textual diagnosis from cardiac device 100 |
| Section 9 | No | Manufacturer | Manufacturer-specific diagnostic and |
| | | Data | overreading data from cardiac device 100 |
| Section 10 | No | Lead | Basic set of measurements and |
| | | Measurement | manufacturer-specific measurements for |
| | | Results | recorded leads |
| Section 11 | No | Universal | Universal statement codes resulting from |
| | | Statement Codes | interpretation by the cardiac device 100 |

With respect to Table 1, Section 0 includes pointers to the start of the remaining sections of the data structure. Section 1 contains information of interest relating to the patient 104 (e.g., the patient's name, age, patient identification number, health status, etc.) and/or to the ECG data (e.g., the acquisition date and type, an identification number for the cardiac device 100 that the patient 104 is wearing, a classification of the type of cardiac device 100 the patient 104 is wearing, etc.). Section 2 contains tables used in encoding the ECG data. For example, the ECG data may be encoded by Huffman coding, and Section 2 may include a Huffman Table used for the encoding. In implementations, the data structure of Table 1 may also include reference data that the cardiac device 100 used to determine that the patient 104 is suspected of experiencing a cardiac arrhythmia (e.g., a baseline ECG taken for the patient 104). As such, Section 2 may also contain encoding tables for the reference data (which is why Section 2 is marked as being dependent). Section 3 indicates which ECG leads are represented in the data structure (e.g., which ECG electrodes 202 were used to generate the ECG signal reflected in the data structure).

If reference data is encoded in the data structure, Section 4 identifies the position of the reference data relative to the ECG signal in the ECG segment (e.g., encoded in Section 6). Section 5 includes the reference data itself. Section 6 includes the encoded ECG data. In implementations, the reference data may be subtracted from the ECG data in the ECG segment, and Section 6 may alternatively or additionally include this “residual” signal. Section 7 contains global measurements, for example, for each reference beat type or QRS complex contained in record.

Section 8 contains the actual text of the diagnostic interpretation made by the cardiac device 100 (e.g., that the cardiac device 100 suspects that the patient 104 is experiencing an arrhythmia, a type of arrhythmia the cardiac device 100 suspects that the patient is experiencing, etc.). Section 9 contains manufacturer-specific diagnostic statements and overreading trails of the interpretation of the ECG data in the ECG segment. For instance, Section 9 may include manufacturer-specific codes related to the interpretation contained in Section 8. Section 10 includes a set of basic measurements and/or manufacturer-specific measurements of the ECG electrodes 202 used to generate the ECG data in the ECG segment. Section 11 includes the interpretation and overreading data according to universal statement codes (e.g., provided by a regulatory body).

As described above, the cardiac device 100 may transmit data to the remote server 102 via cellular networks, via Bluetooth®-to-TCP/IP access point communication, via Wi-Fi, via IoT protocols, etc. For example, in implementations, the cardiac device 100 may transmit the one or more ECG segments to the remote server 102 using MQTT protocols. To illustrate, if the communication link between the cardiac device 100 and the remote server 102 is reliable, or the cardiac device 100 is transmitting lower priority notifications or data to the remote server 102, the cardiac device 100 may transmit the messages using an MQTT Quality of Service (QoS) level 0. At the MQTT QOS level 0, the message is simply transmitted zero or more times. However, for higher priority notifications or data, including the one or more ECG segments, the cardiac device 100 may transmit the message with the one or more ECG segments according to QoS level 1. As such, the cardiac device 100 sends the message one time and then repeatedly until the cardiac device 100 receives a confirmation (e.g., “PUBACK” response) from the remote server 102.

In implementations, the cardiac device 100 may also set a RETAIN flag in the MQTT message to such that the remote server 102 stores the message. Retained messages may also be associated with topics such that, for example, the remote server 102 can transmit retained messages for specific topics to other devices (e.g., a technician interface 106 and/or a caregiver interface 108). To illustrate, retained messages may be associated with specific patients. A caregiver may thus request any retained messages for the patient 104 via the caregiver interface 108, and the remote server 102 may transmitted the retained messages for the patient 104 to the caregiver interface 108. The remote server 102 receives the one or more ECG segments from the cardiac device 100 at step 308. An example process of the cardiac device 100 transmitting the one or more ECG segments to the remote server 102 at step 306 and the remote server 102 receiving the one or more ECG segments at step 308 may be performed as follows.

As an initial matter, a first lightweight protocol (e.g., providing for messages carrying between around 10 MB to around 750 MB of data transfer) can be implemented to transmit messages to the remote server indicating that one or more suspected arrhythmias have been detected in the patient, and/or associated ECG segments, are available for remote server analysis. For example, the first lightweight protocol may carry up to around 128 KB of data, up to around 256 MB of data, and so on. A second protocol that is capable of handling higher bandwidth (e.g., providing for messages greater than 1000 MB, e.g., 750 MB to 1500 MB, or more) can then be implemented to facilitate transfer of the actual underlying ECG segments. For example, the second higher bandwidth protocol may carry up to around 5 GB of data, up to around 5 TB of data (e.g., by using multipart uploads), and so on. With reference to FIG. 13, the cardiac device 100 may send a message to the remote server 102 via the IoT gateway 1306 (e.g., using MQTT protocols such as MQTT v3.1.1 specification from the Organization for the Advancement of Structured Information Standards (OASIS)) indicating that the cardiac device 100 has one or more ECG segments to upload. Additionally or alternatively, such a message may be server via HTTP requests. An advantage of MQTT is based on its lightweight implementation. For example, in some scenarios, the MQTT request can be as small as 2-4 bytes, and avoids header information of the like necessitated in HTTP protocols. For example, an MQTT request can include a topic portion specifying a unique identity of the cardiac device 100, and a payload portion specifying details pertaining to the one or more ECG segments. The IoT gateway 1306 can return an acknowledgment that the MQTT was successfully received.

In response to the MQTT request, the remote server 102 can transmit an address to a predetermined location on the remote server 102 (e.g., a prespecified uniform resource locator (URL) corresponding to the location on the server 102) to the cardiac device 100 that the cardiac device 100 uses to upload the entire one or more ECG segments (e.g., using the HTTP PUT method). The cardiac device 100 may upload the one or more ECG segments to a file database 1308 of the file storage 1300 by uploading the one or more ECG segments to the prespecified URL, or the remote server 102 may retrieve the one or more ECG segments from the URL and store them in the file database 1308 of the file storage 1300.

In implementations, upon receiving the one or more ECG segments, the data record processing 1302 may process the one or more ECG segments to extract elements from the one or more ECG segments, such as a points file containing the data points needed to plot the ECG from the one or more ECG segments, a flags file containing all flag data, and a metadata file. Alternatively or additionally, in implementations, the cardiac device 100 may transmit one or more elements from the one or more ECG segments separately to the remote server 102 (e.g., to the data record processing 1302 and/or mobile data record processing 1304 using MQTT protocols and via the IoT gateway 1306). These separately transmitted one or more elements may include, e.g., a points file, flag file, and/or metadata file. In implementations, the data record processing 1302 may also normalize a data record created for the one or more ECG segments (e.g., with the data record including the extracted elements) and/or perform additional processing of the data record. For example, the data record processing 1302 may further insert into the data record additional fields used in reviewing the data record and insert additional metadata for the data record. The data record processing 1302 may store the extracted elements and/or normalized data record in the search database 1310 (e.g., such that the stored extracted elements and/or normalized data record may be later retrieved by a technician interface 106 and/or a caregiver interface 108).

If the one or more ECG segments include data that the remote server 102 determines should be archived, the remote server 102 may store the data to be archived in a data reservoir 1312 of the mobile data record processing 1304. As an example, the remote server 102 may archive all ECG segments uploaded to the file database 1308 in the data reservoir 1312. As another example, the remote server 102 may archive some or all data records created by the data record processing 1302 in the data reservoir 1312. As another example, the remote server 102 may archive ECG segments and data records that the remote server 102 analyzes to determine whether the patient 104 is experiencing a treatable and/or alertable arrhythmia as part of process 300 in the data reservoir 1312.

Returning to FIG. 3, the remote server 102 analyzes the one or more ECG segments to generate a remote indication as to whether the patient 104 is experiencing a treatable and/or alertable arrhythmia at step 310. In implementations, the remote server 102 may use a machine learning system to determine whether the one or more ECG segments contain, for example, evidence that the patient 104 is experiencing a treatable arrhythmia (e.g., such as ventricular fibrillation) or noise. The machine learning system may be any system of computer algorithms that improve automatically through experience. Suitable machine learning systems may employ unsupervised learning (in which patterns are identified in a stream of input without target labels) or supervised learning (where patterns are identified to match a designated target label). One form of supervised learning is classification, through which an item is categorized based on observation of examples of items from several categories. Another form is numerical or statistical regression, in which a function is developed to describe the relationship between inputs and outputs and to predict how outputs should change as inputs change.

Methods of supervised learning may also include rules-based decision trees, ensemble methods (gabbing, boosting, random forest), the k-Nearest Neighbors algorithm (k-NN), linear or logistic regression, naïve Bayes classifiers, neural networks, the perception algorithm, and the support vector machine (SVM) model. Machine learning classifiers include the MARS™ classifier available from Salford Systems of San Diego, Calif., and the ARESLab™ Adaptive Regression Splines toolbox for Matlab/Octive available from Gints Jekabsons, of the Institute of Applied Computer System, Riga Technical University of Riga, Latvia. A suitable neural network classifier is Neural Network Toolbox™ from The MathWorks, Inc. Classifiers are described in greater detail below.

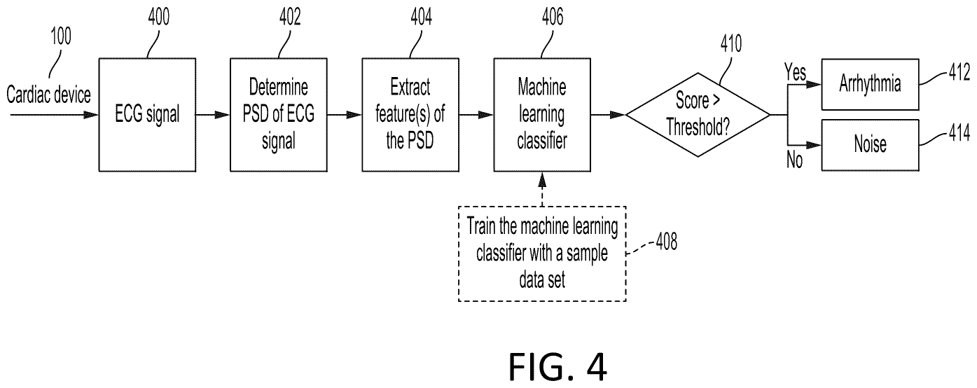

FIG. 4 illustrates a sample process flow the cardiac device 100 may use to perform step 310. As such, the sample process flow can be implemented by at least one processor of the remote server 102. The method shown in FIG. 4 may be for distinguishing a treatable and/or alertable arrhythmia from noise in one or more ECG segments corresponding to a suspected arrhythmia identified by the cardiac device 100. The remote server 102 receives the one or more ECG segments corresponding to a suspected arrhythmia from the cardiac device at block 400. The remote server then 102 determines a power spectral density (PSD) of the one or more ECG segments at block 402.

In implementations, at block 402, the remote server 102 processes the one or more ECG segments to transform the ECG segments. As an illustration, the remote server 102 may transform the time domain ECG segment(s) into frequency domain ECG segment(s). As another illustration, the remote server 102 may transform the one or more ECG segments into a representation of the power distribution of the ECG signal of the one or more ECG segments over a range of frequencies of the ECG signal. In examples, the power distribution over the range of frequencies can be computed from the frequency domain ECG signal. In examples, the transformed ECG signal can be a PSD, which describes how the power of the ECG signal is distributed over the different frequencies of the signal. For example, the remote server 102 may generate the PSD by performing fast Fourier transform (FFT) operations on the time domain ECG signal, or it may employ other discrete Fourier transform (DFT) techniques to generate the PSD.

For example, the PSD may be calculated using a modification of Welch's method. A 4096 sample (at 400 Hz sampling rate) may be windowed into 512 sample windows with a 256 sample overlap. A Hamming window is then applied to each segment. The FFT of each segment is taken, and all of the windows are averaged into a single mean periodogram. The Welch method evaluates FFT, which is a complex number, and then produces the square of the modulus of the FFT to transform the FFT into a real number as a result.

The remote server 102 extracts at least one feature of the PSD and determines an ECG-derived score at block 404. For example, the PSD may have distinct features when a signal that is in ventricular tachycardia (VT)/ventricular fibrillation (VF) is compared with a signal from an ECG demonstrating a normal sinus rhythm, even in the presence of a significant amount of noise contamination. For instance, the PSD of an ECG signal that is in VT/VF may have several distinct dominant spectral bands, while the PSD over an ECG signal that is in VT/VF may have several distinct dominant spectral bands at less than 2.5 Hz. In addition, even inf the presence of a substantial amount of noise, a PSD of a normal sinus rhythm may differ from a PSD of a VT/VF arrhythmia as noise within the ECG signal may be characterized as entropy (e.g., randomness). Accordingly, various entropy calculations may be performed on a PSD to differentiate between a normal sinus rhythm signal with noise and a VT/VF signal. For instance, an in-band entropy may be calculated for a PSD of an ECG signal. In one example, the definition commonly known as the Shannon entropy may be used, and an in-band entropy 61 is the Shannon entropy calculation for frequencies between 2 Hz and 6 Hz. A first-band entropy may also be calculated for a PSD of an ECG signal. The first-band entropy may be calculated by converting the PSD to a probability distribution function (PDF) and calculating the entropy of the signal between 0 Hz and 2 Hz. Finally, the variance of the PSD may be calculated for a PSD of an ECG signal by treating the PSD as a PDF and calculating the second moment, as described in greater detail below.

As such, in implementations, the four features of the PSD may be a dominant frequency of the PSD, an in-band entropy of the PSD between frequencies of 2 Hz and 6 Hz, a first-band entropy of the PSD between frequencies of 0 Hz and 2 Hz, and a variance of the PSD. The remote server 102 may select these features and extract them at block 404, and then submit the extracted features to the machine learning classifier at block 406 based on a combination of feature selection experimentation and physiological reasoning.

For example, in implementations, when a normal sinus rhythm (NSR) in the absence of noise is compared to an NSR contaminated with motion artifact or machine noise, some characteristics of the PSD remain the same. As an illustration, since entropy is a measure of randomness, the entropy in the 0-2 Hz range of the PSD may be similar for an NSR with and without noise. However, the PSD for an NSR without the presence of noise may have much less information content in the 206 Hz range than a PSD for an NSR with a noisy signal. In contrast, a VT/VF ECG signal that is substantially free from noise may have very little information in the 0-2 Hz band and much more information in the 2-6 Hz band of the PSD around the frequency of the VT/VF. Even with noise contamination of the VT/VF signal, most of the frequency content may still be greater than 2 Hz, leaving the entropy for the 0-2 Hz largely untouched. Because the entropies in the 0-2 Hz and 2-6 Hz bands may be different in PSDs representing VT/VF arrhythmias and NSRs with noise contamination, the in-band entropy of the PSD between frequencies of 2 Hz and 6 Hz and first-band entropy of the PSD between frequencies of 0 Hz and 2 Hz may be selected as extracted features, as described above.

To complement the entropy measurements for the two bands (0-2 Hz and 2-6 Hz), the remote server 102 may also investigate the dominant frequency of the PSD. For normal sinus rhythm, the dominant frequency may typically be less than 2 Hz. However, extreme noise content (e.g., either low or high frequency content) may skew this observation. For example, a PSD for an NSR with noise content may have a dominant frequency at about 2.5 Hz and another, less dominant frequency at about 4 Hz. Accordingly, the dominant frequency of the PSD may be selected as an extracted feature, as described above.

Finally, variance of a distribution may provide a feel for the relative spread of the distribution. If a PSD has most of the energy in the 0-2 Hz band, and very little in the 2-6 Hz band, the variance is relatively small. However, a PSD with much energy in the 2-6 Hz band would provide a much wider variance of the PSD. As noted before, a PSD for an NSR may have most of the energy in the 0-2 Hz band, and a PSD for a VT/VF arrhythmia may have more energy in the 2-6 Hz band. In order to calculate variance, it may be assumed that the PSD is a normal distribution. However, in reality, neither the PSD of an NSR signal nor the PSD of a signal in VT/VF may have a normal distribution, so the variance of the PSD may be calculated by treating the PSD as a PDF and calculating the second moment. As such, the second moment calculation may be selected as an extracted feature, as described above.

The remote server 102 determines a predetermined threshold score with a machine learning classifier at block 406 that has been trained with a sample data set at block 408. In implementations, once the dominant frequencies of the PSD, the in-band entropy of the PSD between frequencies of 2 Hz and 6 Hz, the first-band entropy of the PSD between frequencies of 0 Hz and 2 Hz, the variance of the PSD, and/or any other suitable features of the PSD are extracted from the PSD, the remote server 102 may submit these extracted features to the machine learning classifier at block 406. As noted above, any suitable machine learning classifier may be utilized such as, but not limited to, a multivariate adaptive regression splines classifier.

In the following description, the term MARS classifier may be used in a generic sense to describe a multivariate adaptive regression splines classifier. In general, a MARS classifier may be a non-parametric regression classifier that can automatically model non-linearities and interactions between variables. A MARS classifier may thus have the ability to put “kinks” into the regression function. A MARS classifier may build a model that is a weighted sum of basis functions. A basis function may be a constant, a hinge function, or a product of two or more hinge functions. A hinge function may partition data into disjoint independent regions, with a constant (known as a knot) at the hinge of the regions. The product of two or more hinge functions may model interaction between two or more variables.

The MARS classifier may build a model in two phases: a forward pass, followed by a backward pass. In the forward pass, the model starts with an intercept function, adding an intercept term (which is the mean of response values), and then finds basis functions (optimal knot functions) to add to the model. The classifier continues to add basis functions until a residual error becomes too small to overcome. A backward pass is then performed in which the classifier attempts to remove basis functions one-by-one to delete the least effective terms.

As discussed above, the dominant frequency, in-band entropy, first-band entropy, and variance may be mapped using a function derived from a training set that maps each value to a range of −1 to 1. Once these features are mapped, the values may be inputs to the MARS classifier function. As described above, this classifier function is a machine-learning methodology that is trained, tested, and validated using standard machine learning techniques. For example, without limiting the present disclosure, a collection of 120 signals derived from treatments performed by cardiac devices 100 may be stored as a training data set. The training data set may include 60 noisy sinus rhythm signals (i.e., false positive detections) and 60 noisy normal sinus rhythm signals (i.e., false positive detections) and 60 tachyarrhythmia signals. The classifier may have been trained using the training data set described above. The MARS classifier that is utilized in the current example may include two classifiers: one for a side-to-side ECG electrode channel and one for a front-to-back ECG electrode channel. Each classifier may output a numeric value intended to be between 0 and 1.

Any exemplary methodology utilized by the MARS classifier may be described as follows. Once all of the features are extracted from the PSD (denoted by y_{df}, yᵥₐᵣ, y_{H0-2}, y_{H2-6} as discussed above), they may be fed into the MARS classifier function G( ). The MARS classifier function may be a nonlinear function that was generated by examining testing data to determine coefficients that provide a score from 0 to 1 for scaled data within a training data range as discussed hereinabove. The output score is therefore:

$$r = G ( {y}_{df} , {y}_{var} , {y}_{H 0 - 2} , {y}_{H 2 - 6} )$$

This process may be repeated independently for each channel. A continuous 10-second buffer is maintained and the entire process may be repeated in a sliding 10-second window with a 1-second overlap.

Once the cardiac device 100 has determined the ECG-derived score (e.g., r as described above) and the predetermined threshold (e.g., derived based on the training set of data at block 408), the cardiac device 100 classifies the one or more ECG segments as corresponding to a treatable and/or alertable arrhythmia or not based on whether the ECG-derived score is above or below the predetermined threshold at block 410. For example, if the ECG-derived score is one of above or below the predetermined threshold score determined by the machine learning classifier, the remote server 102 provides a remote indication that the patient 104 is experiencing a treatable and/or alertable arrhythmia at block 412, and if the ECG-derived score is the other of above or below the predetermined threshold score, the remote server 102 provides a remote indication that the patient 104 is not experiencing a treatable and/or alertable arrhythmia at block 414. In this way, the remote server 102 may determine whether the one or more ECG segments show that the patient 104 is experiencing a treatable and/or alertable arrhythmia or whether the one or more ECG segments contain noise (e.g., from a malfunction of the ECG electrodes 202, from electromagnetic interference, from patient movement, etc.). Whether the ECG-derived score corresponds to a treatable and/or alertable arrhythmia when the ECG-derived score is above the predetermined threshold depends on the determination of the predetermined threshold from the training set of data. Accordingly, in some implementations, an ECG-derived score above the predetermined threshold generates a remote indication that the patient 104 is not experiencing a treatable and/or alertable arrhythmia, and in some implementations, an ECG-derived score above the predetermined threshold generates a remote indication that the patient 104 is experiencing a treatable and/or alertable arrhythmia.

Alternatively, in various embodiments, the remote server 102 may use a different methodology for analyzing the one or more ECG segments to generate the remote indication. As an example, in implementations, the remote server 102 may analyze the one or more ECG segments to determine whether the patient 104 is experiencing a treatable and/or alertable arrhythmia using a process similar to a process performed by the cardiac device 100, such as the process described below with reference to step 316.

Once the remote server 102 generates a remote indication as to whether the patient is experiencing a treatable and/or alertable arrhythmia, the remote server 102 transmits the remote indication to the cardiac device 100 at step 312. For example, the remote server 102 may transmit the remote indication to the cardiac device 100 using MQTT, HTTP, and/or TCP/IP protocols. In implementations, the remote server 102 may additionally or alternatively transmit the remote indication to a technician interface 106 and/or a caregiver interface 108. For instance, in embodiments, before transmitting a remote indication to the cardiac device 100, the remote server 102 may transmit the remote indication (e.g., along with the associated one or more ECG segments) to a technician interface 106 for a technician to review and confirm whether the technician agrees that the patient 104 is experiencing a treatable and/or alertable arrhythmia. The remote server 102 may transmit the remote indication to the technician interface 106 for all remote indications, or the remote server 102 may transmit a subset of the remote indications to the technician interface 106. As an example, the remote server 102 may not transmit a remote indication to the technician interface 106 if the remote indication indicates that the patient 104 is experiencing a life-threatening cardiac arrhythmia, such as ventricular fibrillation. However, if the remote indication indicates that the patient 104 is experiencing a cardiac arrhythmia unlikely to be life-threatening, such as atrial fibrillation, the remote server 102 may transmit the remote indication to the technician interface 106. As another example, the remote server 102 may transmit the remote indication to the technician interface 106 if the remote server 102 determines that the remote indication is below a predetermined confidence level. In examples, if the remote server 102 does not receive a response from the technician interface 106 within a predetermined amount of response time, the remote server 102 may proceed with transmitting the remote indication to the cardiac device 100.

In embodiments, the remote server 102 may transmit the remote indication (e.g., along with the associated one or more ECG segments) to a caregiver interface 108 associated with a doctor or other clinician for the patient 104 to alert the doctor that the patient 104 appears to be experiencing a treatable and/or alertable arrhythmia. The doctor may, in examples, act like a technician and confirm whether the patient 104 is experiencing the treatable and/or alertable arrhythmia. In embodiments, the remote server 102 may transmit the remote indication to an at-home caregiver of the patient 104 to alert the at-home caregiver that the patient 104 appears to be experiencing a treatable and/or alertable arrhythmia (e.g., such that the at-home caregiver may need to provide or seek medical help for the patient 104). In embodiments, the remote server 102 may transmit the remote indication to a caregiver interface 108 associated with a family member or loved one of the patient 104 to provide a warning the patient 104 appears to be experiencing a treatable and/or alertable arrhythmia.

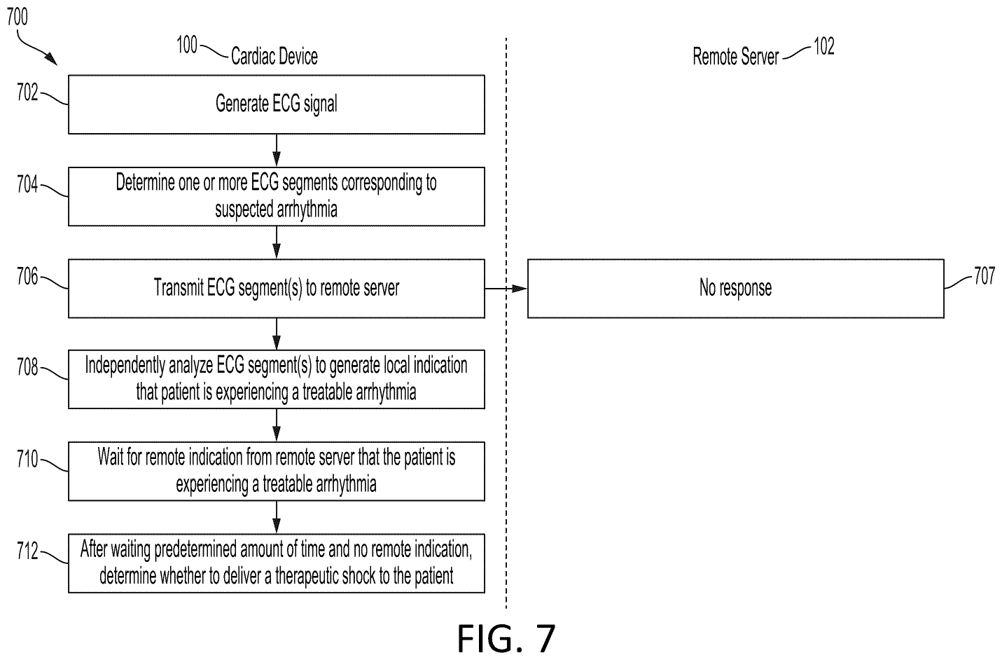

The cardiac device 100 receives the remote indication, which as described above is based on the one or more ECG segments, as to whether the patient 104 is experiencing a treatable and/or alertable arrhythmia at step 314. The cardiac device 100 also independently analyzes the one or more ECG segments to generate a local indication as to whether the patient 104 is experiencing a treatable and/or alertable arrhythmia at step 318. In implementations, the cardiac device 100 performs the independent analysis of the one or more ECG segments at the same time the remote server 102 is analyzing the ECG segments to determine whether the patient 104 is experiencing a treatable and/or alertable arrhythmia (e.g., immediately after, or concurrent to, transmitting the one or more ECG segments to the remote server 102 at step 306). As such, the cardiac device 100 may perform the independent analysis of the one or more ECG segments while waiting for the remote indication from the remote server 102. In implementations, the cardiac device 100 may be configured to wait a predetermined amount of time for the remote indication after generating the local indication. For example, the cardiac device 100 may be configured to wait a predetermined amount of time for the remote indication upon generating a local indication that the ambulatory patient is experiencing a treatable and/or alertable arrhythmia (e.g., after which, the cardiac device 100 proceeds to the process described below with respect to FIG. 7).

In implementations, the cardiac device 100 performs steps 304 and 316 simultaneously. For example, the cardiac device 100 may generate the local indication as to whether the patient 104 is experiencing a treatable and/or alertable arrhythmia as part of determining one or more ECG segments corresponding to the suspected arrhythmia.

Alternatively, in implementations, the cardiac device 100 performs steps 304 and 316 using different processes. For instance, the cardiac device 100 may be configured to determine the one or more ECG segments corresponding to a suspected arrhythmia occurring in the patient 104 based on the ECG signal using a first process and independently analyze the one or more ECG segments to generate a local indication as to whether the patient 104 is experiencing a treatable and/or alertable arrhythmia using a second process. In examples, the first process may be less computationally intensive and/or require lower power than the second process. As such, the cardiac device 100 may only use the second more computationally intensive and/or power-consuming process when the cardiac device 100 already suspects that the patient 104 is experiencing a treatable and/or alertable arrhythmia. In implementations, the cardiac device 100 may perform step 316 similarly to how the remote server 102 analyzes the one or more ECG segments to generate a remote indication as to whether the patient 104 is experiencing a treatable and/or alertable arrhythmia at step 310.

In implementations, the cardiac device 100 may perform the independent analysis of the one or more ECG segments before transmitting the one or more ECG segments corresponding to the suspected arrhythmia to the remote server 102. As an illustration, the cardiac device 100 may perform steps 304 and 316 (e.g., using the same or different processes, as described above) before transmitting the one or more ECG segments to the remote server 102 at step 306. Thus, the cardiac device 100 may query the remote server 102 for the remote indication upon generating the local indication. For example, the query may include the one or more ECG segments that are transmitted to the remote server 102. Alternatively, the remote server 102 may perform the independent analysis of the one or more ECG segments to generate the local indication while waiting for the remote indication from the remote server 102, as described above. If the cardiac device 100 generates a local indication that the patient 104 is experiencing a treatable and/or alertable arrhythmia, the cardiac device 100 may query the remote server 102 for the remote indication (e.g., if the remote server 102 has not already provided the remote indication). The query may include the one or more ECG segments already transmitted to the remote server 102 and/or one or more additional ECG segments (e.g., ECG segments from another channel of the ECG electrodes 202, more current ECG segments, etc.).

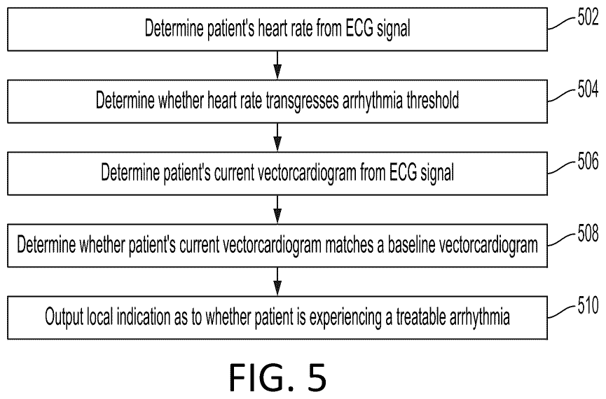

FIG. 5 illustrates a sample process flow the cardiac device 100 may use to perform step 316. As such, the sample process flow shown in FIG. 5 can be implemented by the medical device controller 206 of the cardiac device 100. The medical device controller 206 determines the patient's heart rate from the ECG signal at step 502. For example, the medical device controller 206 may use a QRS detector on the ECG signal to determine the patient's heart rate. As another example, the medical device controller 206 may determine the patient's heart rate by performing a fast Fourier transform (FFT) on the ECG signal or signals, with the FFT decomposing the analog ECG waveform into its frequency components. The medical device controller 206 may then analyze the output of the FFT to determine the strongest frequency component indicative of heart rate. In implementations, as described above with reference to step 302 of process 300, the medical device controller 206 may generate more than one ECG signal (e.g., with each ECG signal corresponding to a different channel of the ECG electrodes 202). The medical device controller 206 may thus use a QRS detector and analyze a FFT on each ECG signal to provide multiple independent assessments of the patient's heart rate.

In implementations, the medical device controller 206 may use multiple methods to determine the patient's heart rate, for example, weighting the outputs of the methods to produce a final measure of the patient's heart rate. As an illustration, the medical device controller 206 may apply logical weights based on comparing ECG channels, signal quality, and historic heart rate values to determine the best inputs for accurately monitoring the patient's heart rate. For example, if the heart rate from QRS detectors used on multiple ECG channels do not match, the medical device controller 206 may apply less weight to these inputs and greater weight to other sources.

Once the medical device controller 206 has determined the patient's heart rate, the medical device controller 206 then determines if the patient's heart rate transgresses an arrhythmia threshold at step 504. For example, the medical device controller 206 may determine if the patient's heart rate transgresses a threshold generally used for arrhythmias (e.g., 150, 160, 170, etc. bpm). As another example, the medical device controller 206 may determine if the patient's heart rate transgresses a threshold used for a specific arrhythmia. To illustrate, the medical device controller 206 may determine if the patient's heart rate is below a threshold for ventricular tachycardia, above the threshold for ventricular tachycardia but below a threshold for ventricular fibrillation, or above the threshold for ventricular fibrillation. As another illustration, the medical device controller 206 may determine if the patient's heart rate is at or below a threshold for bradycardia, at or above a threshold for atrial fibrillation, at or above a threshold for ventricular tachycardia, and/or at or above a threshold for ventricular fibrillation. In implementations, these thresholds may be programmed for the patient 104 (e.g., during a setup period).

The medical device controller 206 determines the patient's current vectorcardiogram from the ECG signal at step 506. For example, the ECG sensors 202 may be positioned around the patient's torso when the patient is wearing the cardiac device 100 to form orthogonal leads (e.g., front-to-back and side-to-side at the level of the patient's xiphoid process). The medical device controller 206 may determine a direction and magnitude of the electrical forces in the patient's heart and plot them (e.g., on an x-y or an x-y-z graph) to form a vectorcardiogram. In implementations, the medical device controller 206 may determine the patient's vectorcardiogram if the patient's heart rate transgresses an arrhythmia threshold at step 504. In implementations, the medical device controller 206 may determine the patient's current vectorcardiogram independent of whether the patient's heart rate transgresses an arrhythmia threshold at step 504. In implementations, the medical device controller 206 may determine the patient's current vectorcardiogram as part of determining the patient's heart rate from the ECG signal. For instance, the medical device controller 206 may plot the patient's current vectorcardiogram, determine the amount of time that it takes for the vectorcardiogram to repeat (e.g., for the plot to return to a starting point of within a certain vicinity of the starting point), and use that determination to output a heart rate for the patient.

The medical device controller 206 determines whether the patient's current vectorcardiogram matches a baseline vectorcardiogram at step 508. To illustrate, the medical device controller 206 may take a baseline vectorcardiogram for the patient 104 during a setup period and/or periodically during the patient's use of the cardiac device 100 (e.g., weekly at a predetermined time, after the patient 104 is delivered a therapeutic shock, etc.). The medical device controller 206 may then compare the patient's current vectorcardiogram to the patient's baseline vectorcardiogram to determine if the two morphologies match with a predetermined degree of accuracy. In implementations, the predetermined degree of accuracy may vary for the different types of arrhythmias that the medical device controller 206 can detect. If the patient's current vectorcardiogram does not match their baseline vectorcardiogram, this failure to match may serve as evidence that the patient 104 is experiencing a treatable and/or alertable arrhythmia. If the patient's current vectorcardiogram does match their baseline vectorcardiogram with the predetermined degree of accuracy, this match may serve as evidence that the patient 104 is not experiencing a treatable and/or alertable arrhythmia. In implementations, the medical device controller 206 may determine whether the patient's current vectorcardiogram matches their baseline vectorcardiogram only if the medical device controller 206 has already determined that the patient's heart rate transgresses an arrhythmia threshold at step 504. In implementations, the medical device controller 206 may not use the patient's vectorcardiogram morphology, for example, if the signal quality from one of the ECG sensors 202 is unreliable or if the patient's heart rate is above the ventricular fibrillation threshold. In such cases, the medical device controller 206 may instead rely primarily on heart rate, stability (e.g., whether the R-R intervals of the patient's heart rate are consistent or inconsistent), onset criteria (e.g., whether the patient has experienced rapid changes in heart rate), and/or the like.

The medical device controller 206 outputs a local indication as to whether the patient 104 is experiencing a treatable and/or alertable arrhythmia at step 510. For example, if the patient's heart rate does not transgress an arrhythmia threshold, the medical device controller 206 may output a local indication that the patient 104 is not experiencing a treatable and/or alertable arrhythmia. As another example, if the patient's heart rate transgresses an arrhythmia threshold but the patient's current vectorcardiogram matches their baseline vectorcardiogram with the predetermined degree of accuracy, the medical device controller 206 may also output a local indication that the patient 104 is not experiencing a treatable and/or alertable arrhythmia. As another example, if the patient's heart rate transgresses an arrhythmia threshold and the patient's current vectorcardiogram fails to match their baseline vectorcardiogram, the medical device controller 206 may output a local indication that the patient 104 is experiencing a treatable and/or alertable arrhythmia. As another example, if the patient's heart rate transgresses a first arrhythmia threshold (e.g., a threshold for ventricular fibrillation) but not a second arrhythmia threshold (e.g., a threshold for ventricular tachycardia that is higher than the threshold for ventricular fibrillation), and the patient's current vectorcardiogram fails to match their baseline vectorcardiogram, the medical device controller 206 may output a local indication that the patient 104 is experiencing a treatable arrhythmia. As another example, if the patient's heart rate transgresses an arrhythmia threshold corresponding to a non-treatable cardiac arrhythmia (e.g., a threshold for ventricular tachycardia), the medical device controller 206 may output a local indication that the patient 104 is experiencing a non-treatable arrhythmia even if the patient's current vectorcardiogram fails to match their baseline vectorcardiogram.

In implementations, the medical device controller 206 applies a confidence level as part of determining whether the patient is experiencing a treatable and/or alertable arrhythmia. As such, the medical device controller 206 may assign weights to various inputs, such as the patient's heart rate, vectorcardiogram morphology, response button use (e.g., whether the patient 104 has already been alerted to a treatable and/or alertable arrhythmia and used a response button on the patient interface pod 210), signal quality, and/or the like to determine a confidence level for whether the patient is experiencing a treatable and/or alertable arrhythmia. The weighted inputs can contribute positively or negatively to the confidence level. If the medical device controller 206 determines that an input is unreliable (e.g., signal quality is unreliable, different methods of determining the patient's heart rate produce different heart rate measures, etc.), the medical device controller 206 may decrease the weight for that input. The medical device controller 206 outputs a local indication that the patient 104 is experiencing a treatable and/or alertable arrhythmia if the confidence level transgresses a predetermined confidence level threshold and otherwise outputs a local indication that the patient is not experiencing a treatable and/or alertable arrhythmia.

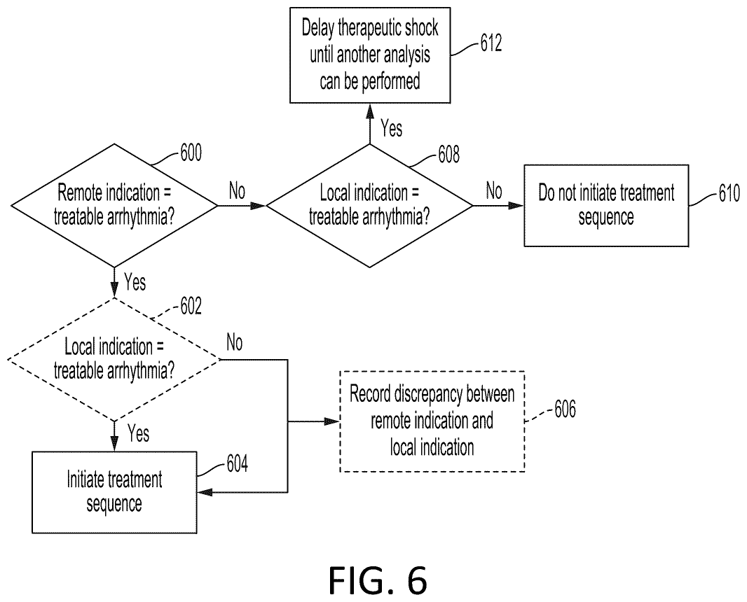

Returning to process 300 of FIG. 3, once the cardiac device 100 has generated the local indication as to whether the patient 104 is experiencing a treatable and/or alertable arrhythmia and received the remote indication from the remote server 102 as to whether the patient is experiencing a treatable and/or alertable arrhythmia, the cardiac device determines whether to deliver a therapeutic shock to the patient 104 based on the remote indication and the local indication at step 318. In implementations, the cardiac device is configured to determine whether the remote indication and the local indication indicate that the patient 104 is experiencing a treatable and/or alertable arrhythmia and/or whether the remote indication and the local indication agree as to whether the patient 104 is experiencing a treatable and/or alertable arrhythmia. FIG. 6 illustrates an example process flow that the cardiac device 100 may use as part of determining whether to deliver a therapeutic shock to the patient 104 at step 318.

As shown in FIG. 6, the cardiac device 100 determines whether the remote indication indicates that the patient 104 is experiencing a treatable and/or alertable arrhythmia at step 600. In implementations, if the remote indication does indicate that the patient 104 is experiencing a treatable and/or alertable arrhythmia, the cardiac device 100 determines whether the local indication indicates that the patient 104 is experiencing a treatable and/or alertable arrhythmia at step 602. In response to determining that the local indication also indicates that the patient 104 is experiencing a treatable and/or alertable arrhythmia, the cardiac device 100 initiates a treatment sequence at step 604. For example, the cardiac device 100 may activate an alarm warning the patient 104 that the cardiac device 100 is going to deliver a therapeutic shock. The patient 104 may be able to delay or abort the therapeutic shock by pressing a response button or response buttons on the patient interface pod 210. However, if the patient 104 does not press the response button or buttons, the cardiac device 100 may proceed with delivering the therapeutic shock via the therapy electrodes 204. As another example, the cardiac device 100 may activate an alarm telling bystanders to begin performing CPR on the patient 104. As another example, the cardiac device 100 may activate an alarm telling the patient 104 to retire to a safe local and call for emergency care.

In implementations, if the remote indication indicates that the patient 104 is experiencing a treatable and/or alertable arrhythmia, but the local indication does not indicate that the patient 104 is experiencing a treatable and/or alertable arrhythmia, the cardiac device 100 may still initiate the treatment sequence at step 604. The cardiac device 100 may also record a discrepancy between the remote indication and the local indication at step 606. The cardiac device 100 may later analyze the discrepancy to identify a source of the discrepancy (e.g., differences in how the remote indication and local indication were generated, an update that needs to be made to the process used to generate the local indication, etc.). Alternatively, or additionally, the cardiac device 100 may transmit the discrepancy to the remote server 102 for the remote server 102 to identify the source of the discrepancy. In response to identifying the source of the discrepancy, the cardiac device 100 and/or the remote server 102 may take one or more responsive actions. For instance, the remote server 102 may transmit an update to the cardiac device 100, where the update alters the process the cardiac device 100 will use to identify suspected arrhythmias and/or treatable or alertable arrhythmias in the future.

As a more specific example, the cardiac device 100 and/or the remote server 102 may identify one or more patient-specific idiosyncrasies (e.g., characteristics of the patient's heart rate, vectorcardiogram morphology, the types of arrhythmias the patient 104 has been experiencing, etc.) from the remote and local indications and how the remote and local indications were generated. The remote server 102 may then transmit an update to the cardiac device 100 to adapt the process the cardiac device 100 uses to identify suspected arrhythmias and/or treatable and/or alertable arrhythmias that account for the patient's idiosyncrasies. To illustrate, the update may make the process more sensitive to certain types of arrhythmias that the patient 104 has been experiencing, help make the process of identifying treatable and/or alertable arrhythmias more accurate to the patient 104, etc.

In implementations, steps 602 and 606 may be optional (as shown by the dashed lines in FIG. 6), and the cardiac device 100 may proceed with initiating the treatment sequence in response to determining that the remote indication does indicate that the patient 104 is experiencing a treatable and/or alertable arrhythmia. Alternatively, in implementations, the cardiac device 100 may use a different process after determining whether the local indication indicates that the patient 104 is experiencing a treatable and/or alertable arrhythmia at step 602. For example, if the cardiac device 100 determines that the remote indication indicates that the patient 104 is experiencing a treatable and/or alertable arrhythmia, but that the local indication does not indicate that the patient 104 is experiencing a treatable and/or alertable arrhythmia, the cardiac device 100 may delay the initiation of the treatment sequence to perform a confirmation of whether the patient 104 is experiencing the treatable and/or alertable arrhythmia. As an illustration, the cardiac device 100 may transmit one or more additional ECG segments (e.g., one or more current ECG segments and/or one or more ECG segments from another ECG channel) and/or other physiological data for the patient 104 (e.g., motion data, cardiac vibrational data, posture data, etc.) to the remote server 102. The cardiac device 100 may then perform a second analysis of whether the patient 104 is experiencing a treatable and/or alertable arrhythmia and wait for an additional one or more remote indications regarding whether the patient 104 is experiencing a treatable and/or alertable arrhythmia.

If a local indication and a remote indication generated during the confirmation process agree that the patient 104 is experiencing a treatable and/or alertable arrhythmia, the cardiac device 100 may initiate the treatment sequence at step 604. Alternatively, if a local indication and a remote indication generated during the confirmation process now agree that the patient 104 is not experiencing a treatable and/or alertable arrhythmia, the cardiac device 100 may avoid initiating a treatment sequence and providing any therapeutic shocks to the patient 104. However, if the remote indication generated during the confirmation process continues to indicate that the patient 104 is experiencing the treatable and/or alertable arrhythmia, and the local indication generated during the confirmation process continues to indicate that the patient 104 is not experiencing the treatable and/or alertable arrhythmia, and a predetermined amount of time passes, the cardiac device 100 may decide to proceed with initiating the treatment sequence at step 604. In implementations, this confirmation process may be similar to the second analysis performed at step 612 and described below.

Alternatively, if the cardiac device 100 determines at step 600 that the remote indication does not indicate that the patient 104 is experiencing a treatable and/or alertable arrhythmia, the cardiac device 100 determines whether the local indication indicates that the patient 104 is experiencing a treatable and/or alertable arrhythmia at step 608. If the local indication also indicates that the patient 104 is not experiencing a treatable and/or alertable arrhythmia, the cardiac device 100 does not initiate a treatment sequence at step 610 and avoids delivering a therapeutic shock to the patient 104. Instead, the cardiac device 100 returns to monitoring the patient 104 for suspected arrhythmias. However, if the local indication does indicate that the patient 104 is experiencing a treatable and/or alertable arrhythmia, the cardiac device 100 delays providing a therapeutic shock to the patient 104 until another analysis of the patient's ECG signal can be performed at step 612.

As an illustration, the cardiac device 100 may an initiate a process similar to the confirmation process described above with reference to steps 600, 602, 604, and 606. The cardiac device 100 may transmit one or more additional ECG segments corresponding to the suspected arrhythmia to the remote server 102. For example, the one or more initially-transmitted ECG segments may have been from a first channel of the ECG electrodes 202, and the one or more additional ECG segments may be from a second channel of the ECG electrodes 202. As another example, the initially-transmitted one or more ECG segments may have been from a first period of time, and the one or more additional ECG segments may be from a second period of time later the first period of time (e.g., the one or more additional ECG segments may be segments from the patient's current ECG signal). As another example, the cardiac device 100 may transmit non-ECG data to the remote server 102, such as motion data, cardiac vibrational data, posture data, and/or the like. The cardiac device 100 may also perform a second analysis of the patient's ECG signal. The second analysis may be of the originally-analyzed one or more ECG segments and/or of one or more additional ECG segments corresponding to the suspected arrhythmia (e.g., ECG segment(s) from another ECG channel of the ECG electrodes 202 and/or more ECG segment(s) from a second period of time that are more current). Based on the second analysis, the cardiac device 100 may generate a second local indication as to whether the patient 104 is experiencing a treatable and/or alertable arrhythmia. The cardiac device 100 may also receive a second remote indication as to whether the patient 104 is experiencing a treatable and/or alertable arrhythmia.

If the second remote indication and the second local indication agree that the patient 104 is not experiencing a treatable arrhythmia, the cardiac device 100 may not initiate a treatment sequence at 610. If instead the second remote indication and second local indication agree that the patient 104 is experiencing a treatable arrhythmia, the cardiac device 100 may initiate a treatment sequence for the patient 104. If the second remote indication continues to indicate that the patient 104 is not experiencing a treatable arrhythmia, but the second local indication continues to indicate that the patient 104 is experiencing a treatable arrhythmia, the cardiac device 100 may again delay a therapeutic shock to perform a third analysis. In implementations, the cardiac device 100 may continue this cycle until the remote indication and the local indication agree as to whether the patient 104 is experiencing a treatable arrhythmia or a predetermined amount of delay time has passed. If the predetermined amount of delay time has passed, the cardiac device 100 may proceed to initiate the treatment sequence. In implementations, the cardiac device 100 may instead defer to the remote indication if the remote indication and the local indication disagree as to whether the patient 104 is experiencing a treatable arrhythmia after the second analysis performed at step 612.

In implementations, instead of the cardiac device 100 transmitting specific ECG segments corresponding to a suspected arrhythmia in the patient 104, the cardiac device 100 may continuously transmit ECG data (and, in embodiments, other physiological data such as motion data) to the remote server 102. The remote server 102 and the cardiac device 100 may independently continuously analyze the ECG data for treatable cardiac arrhythmias in the patient 104. If the remote server 102 detects a treatable cardiac arrhythmia, the remote server 102 may transmit the remote indication to the cardiac device 100, and vice versa. Receiving a local indication from the cardiac device 100 may, for example, prompt the remote server 102 to re-analyze recent ECG data for cardiac arrhythmias (if the remote server 102 has not already generated a remote indication indicating that the patient 104 is experiencing an arrhythmia). Similarly, receiving a remote indication from the remote server 102 may prompt the cardiac device 100 to re-analyze recent ECG data, if the cardiac device 100 has not already generated a local indication indicating that the patient 104 is experiencing a cardiac arrhythmia. If the cardiac device 100 has already generated a local indication to the same, the cardiac device 100 may instead initiate a treatment sequence for the treatable arrhythmia.

In implementations, the cardiac device 100 and/or the remote server 102 may identify non-treatable cardiac arrhythmias in addition to treatable cardiac arrhythmias. For example, if the cardiac device 100 and/or the remote server 102 identify that the patient 104 is experiencing a non-treatable cardiac arrhythmia, the cardiac device 100 and/or remote server 102 may store the ECG data for the arrhythmia and a flag indicating the non-treatable cardiac arrhythmia. The remote server 102 may also, for instance, transmit a notification to a caregiver interface 108 indicating that the patient 104 is experiencing a non-treatable cardiac arrhythmia.

FIG. 7 illustrates another sample process flow for remote arrhythmia detection/verification and treatment. The sample process 700 can be implemented by the cardiac device 100 (e.g., the medical device controller 206), as shown in FIG. 7. The cardiac device 100 generates an ECG signal based on the sensed electrical signals indicative of the cardiac activity of the patient at step 702. For example, the ECG signal may be a single channel ECG signal (e.g., based on one or more ECG electrodes), two channel ECG signal (e.g., based on 3 or more ECG electrodes), and/or three or more channel ECG signal (e.g., based on 3 or more ECG electrodes). The cardiac device 100 determines one or more ECG segments corresponding to a suspected arrhythmia occurring in the patient 104 based on the ECG signal at step 704. For example, the ECG segment may include corresponding single channel ECG segment, or two or more channel ECG segments. The cardiac device 100 transmits the determined one or more ECG segments (e.g., 1-channel ECG segments, 2-channel ECG segments, or 3 or more channel ECG segments, as appropriate) corresponding to the suspected arrhythmia occurring in the patient 104 to the remote server 102 at step 706, to which there is no response at step 707. The cardiac device 100 also independently analyzes the one or more ECG segments to generate a local indication that the patient 104 is experiencing a treatable and/or alertable arrhythmia at step 708. In implementations, steps 702, 704, 706, and 708 may be performed similarly to the processes described above with respect to steps 302, 304, 306, and 316, respectively, of FIG. 3. It is noted that steps 706 and 708 need not be performed in the order illustrated in FIG. 7. For example, the cardiac device 100 can independently analyze the ECG segment(s), step 708, before transmitting the associated ECG segment(s) to the remote server, step 706.

Upon generating the local indication that the patient 104 is experiencing a treatable and/or alertable arrhythmia, the cardiac device 100 waits for a remote indication from the remote server 102 that the patient 104 is experiencing a treatable and/or alertable arrhythmia (e.g., with the remote indication described above with reference to steps 308-314 of FIG. 3) at step 710. In implementations, the cardiac device 100 is configured to wait a predetermined amount of time from the determination of the one or more ECG segments corresponding to the suspected arrhythmia at step 704. In implementations, the cardiac device 100 is configured to wait a predetermined amount of time from the generation of the local indication that the patient 104 is experiencing a treatable and/or alertable arrhythmia. The predetermined amount of time may be a default amount of time, or the predetermined amount of time may be configurable (e.g., configurable by a technician input, by a caregiver input, etc.). In implementations, the predetermined amount of time may depend on the type of treatable and/or alertable arrhythmia that the cardiac device 100 has determined that the patient 104 is experiencing. For instance, the predetermined amount of time may be a first period of time if the treatable and/or alertable arrhythmia is ventricular fibrillation, a second period of time if the treatable and/or alertable arrhythmia is bradycardia, and a third period of time if the treatable and/or alertable arrhythmia is atrial fibrillation, where the first period of time is the shortest and the third period of time is the longest. For example, the predetermined amount of times may range from between around (e.g., within a certain amount or percentage, such as 5-20%) 5 seconds to around 60 seconds, between around 20 seconds to around 90 seconds, from between around 45 seconds to around 120 seconds, from between around 60 seconds to around 3 minutes, from between around 1.5 minutes to around 5 minutes, from between around 3 minutes to around 10 minutes. As noted, a physician, caregiver, technician, or other authorized user may also set a user-defined value for the predetermined amount of time.

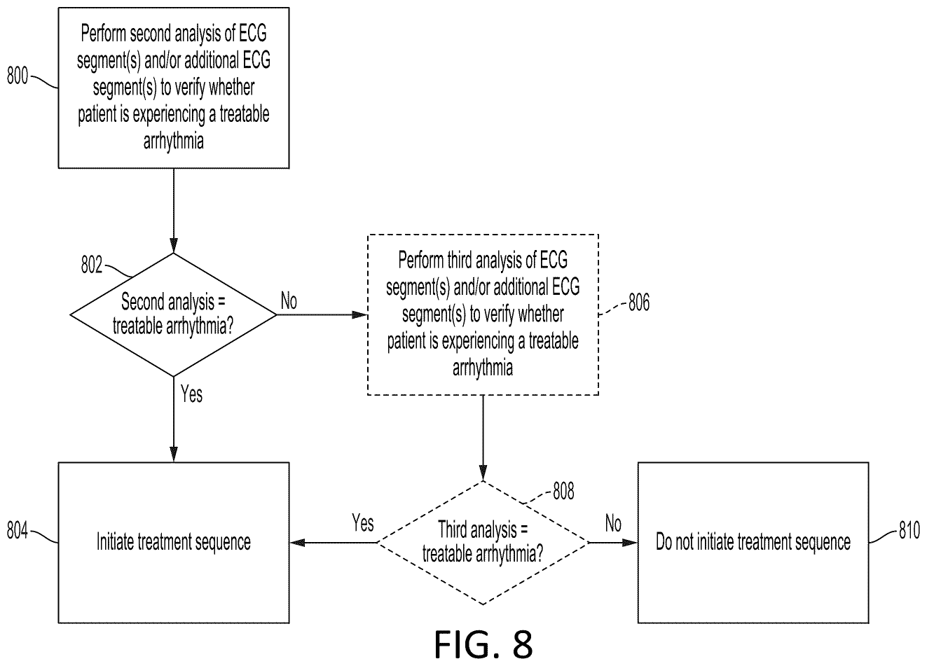

After waiting a predetermined amount of time and receiving no remote indication as to whether the patient 104 is experiencing a treatable and/or an alertable arrhythmia (e.g., VF, VT, bradycardia, tachycardia, asystole), the cardiac device 100 determines whether to deliver, via the therapy electrodes 204, a therapeutic shock to the patient 104 at step 712. FIG. 8 illustrates a sample process flow the cardiac device 100 may use to perform step 712. As such, the sample process flow shown in FIG. 8 can be implemented by the medical device controller 206 of the cardiac device 100. The cardiac device 100 performs a second analysis of the one or more original ECG segments and/or one or more additional, second ECG segments to verify whether the patient 104 is experiencing a treatable and/or alertable arrhythmia at step 800 (e.g., to generate a second local indication as to whether the patient 104 is experiencing a treatable and/or alertable arrhythmia). In implementations, the one or more second ECG segments include one or more ECG segments from a different channel of the ECG electrodes 202 than used for the original ECG segment(s). In implementations, the one or more second ECG segments are from a second period of time compared to the time corresponding to the original ECG segment(s) (e.g., the one or more second ECG segments are more current).

In implementations, the cardiac device 100 performs the second analysis similarly to how the cardiac device 100 independently analyzes the one or more ECG segments to generate a local indication that the patient 104 is experiencing a treatable and/or alertable arrhythmia at step 708 of FIG. 7. In implementations, the second analysis is different from that first analysis. For instance, in the second analysis, the cardiac device 100 may apply a more specific arrhythmia detection process corresponding to the type of treatable and/or alertable arrhythmia the cardiac device 100 determined that the patient 104 is experiencing. As another example, in the second analysis, the cardiac device 100 may additionally use non-ECG data (e.g., such as motion data, cardiac vibrational data, posture data, etc.) as part of determining whether the patient 104 is experiencing a treatable and/or alertable arrhythmia. To illustrate, the cardiac device 100 may use motion data to determine whether the patient 104 is moving (e.g., which may indicate that the patient 104 is not experiencing a treatable and/or alertable arrhythmia), cardiac vibrational data as another source of the heart rate of the patient 104, posture data to determine whether the patient 104 is upright or supine (e.g., where being supine may indicate that the patient 104 is experiencing a treatable and/or alertable arrhythmia), and/or the like.

The cardiac device 100 uses the second local indication output by the second analysis to determine whether the cardiac device 100 has confirmed that the patient 104 is experiencing a treatable and/or alertable arrhythmia at step 802. If the second local indication confirms that the patient is experiencing a treatable and/or alertable arrhythmia, the cardiac device 100 initiates a treatment sequence at step 804. In implementations, the cardiac device 100 may perform step 804 similarly to how the cardiac device 100 performs step 604 of FIG. 6, as described above. However, if the second local indication indicates that that patient 104 is not experiencing a treatable and/or alertable arrhythmia, the cardiac device 100 performs a third analysis of the one or more ECG segments (e.g., the original ECG segment(s) and/or the second ECG segment(s)) and/or one or more additional, third ECG segments to verify whether the patient 104 is experiencing a treatable and/or alertable arrhythmia (e.g., generate a third local indication as to whether the patient 104 is experiencing a treatable and/or alertable arrhythmia). As with the second ECG segment(s), the one or more third ECG segments may be from another ECG channel of the ECG electrodes 202 and/or from a third period of time compared to the first period of time for the original ECG segment(s) and the second period of time for the second ECG segment(s) (e.g., the third ECG segment(s) may be more current than any of the previously-analyzed one or more ECG segments).

In implementations, the third analysis may be similar to the original and/or second analysis used to determine whether the patient 104 is experiencing a treatable and/or alertable arrhythmia. In implementations, the third analysis may be different from the original and/or second analysis used to determine whether the patient 104 is experiencing a treatable and/or alertable arrhythmia. For example, the third analysis may be more specific to the type of treatable and/or alertable arrhythmia the cardiac device 100 has determined the patient 104 is experiencing and/or may use non-ECG data, as similarly described above with reference to the second analysis performed at step 800.

The cardiac device 100 uses the third local indication output by the third analysis to determine whether the cardiac device 100 has confirmed that the patient 104 is experiencing a treatable and/or alertable arrhythmia at step 808. If the third local indication does not indicate that the patient 104 is experiencing a treatable and/or alertable arrhythmia, the cardiac device may not initiate the treatment sequence (e.g., delaying or aborting delivering the therapeutic shock to the patient 104) at step 810. If the third local indication instead indicates that the patient 104 is experiencing a treatable and/or alertable arrhythmia, the cardiac device may initiate a treatment sequence at step 804. Alternatively, in implementations, the cardiac device 100 may continue re-analyzing ECG segments until the cardiac device 100 outputs two consecutive local indications that agree as to whether the patient 104 is experiencing a treatable and/or alertable arrhythmia (or a predetermined amount of time has passed, after which the cardiac device 100 will proceed with initiating a treatment sequence). If the two consecutive local indications indicate that the patient 104 is experiencing a treatable and/or alertable arrhythmia, the cardiac device 100 may then initiate a treatment sequence. If the two consecutive local indications indicate that the patient 104 is not experiencing a treatable and/or alertable arrhythmia, the cardiac device 100 may then abort initiating a treatment sequence.

In implementations, steps 806 and 808 may be optional (as shown by the dashed lines in FIG. 8). Instead, the cardiac device 100 may perform the second analysis and determine whether to initiate a treatment sequence for the patient 104 based on whether the second local indicate indicates that the patient 104 is experiencing a treatable and/or alertable arrhythmia. In implementations, the cardiac device 100 may not perform a second or third analysis at all. Instead, the cardiac device 100 may initiate a treatment sequence if the local indication indicates that the patient 104 is experiencing a treatable and/or alertable arrhythmia and the cardiac device 100 does not receive the remote indication after waiting the predetermined amount of time at step 712 of FIG. 7.

In implementations, if while executing the process shown in FIG. 8, the cardiac device 100 receives a remote indication from the remote server 102, the cardiac device 100 may abort the processes shown in FIGS. 7 and 8. Instead, for example, the cardiac device 100 may return to following the processes shown in FIGS. 3-6 and described above.

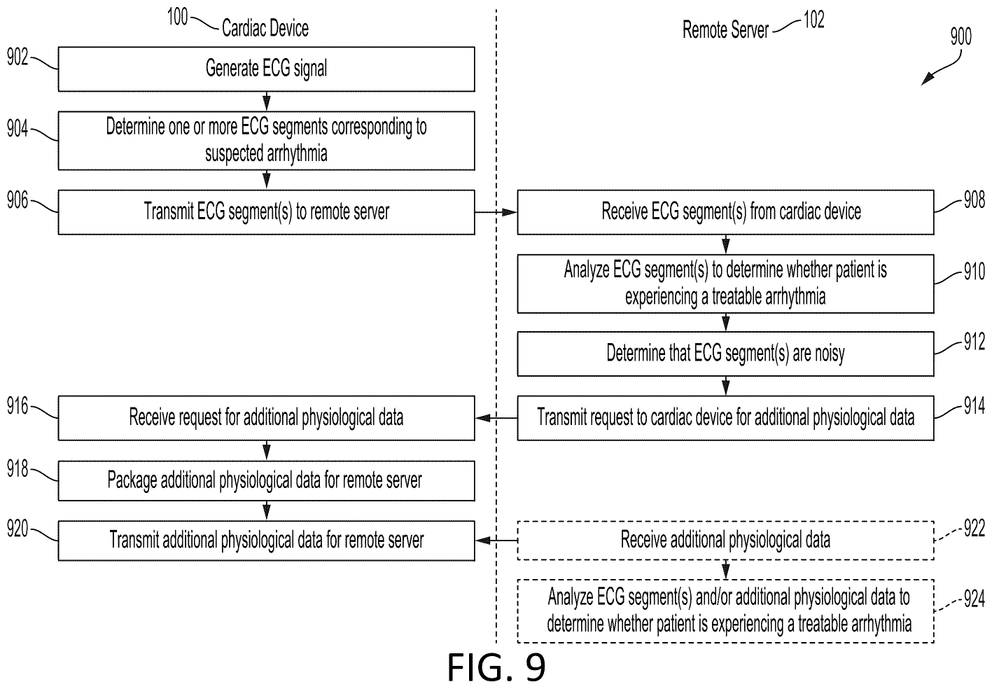

a illustrates a sample process flow for remote arrhythmia detection/verification. The sample process 900 can be implemented by the cardiac device 100 (e.g., the medical device controller 206) and by the remote server 102 in communication with the cardiac device 100 (e.g., at least one processor of the remote server), as shown in FIG. 9. The cardiac device 100 generates an ECG signal at step 902. The cardiac also determines one or more ECG segments corresponding to a suspected arrhythmia at step 904 and transmits the one or more ECG segments corresponding to the suspected arrhythmia to the remote server 102 at step 906. In implementations, steps 902, 904, and 906 may be performed similarly to the processes described above with respect to step 302, 304, and 306, respectively, of FIG. 3.

The remote server 102 receives the one or more ECG segments corresponding to the suspected arrhythmia occurring in the patient from the cardiac device 100 at step 908. The remote server 102 analyzes the received one or more ECG segments to determine whether the patient 104 is experiencing a treatable and/or alertable arrhythmia at step 910. In implementations, steps 908 and 910 may be implemented similarly to the processes described above with respect to step 308 and 310 of FIG. 3. However, the cardiac device 100 determines that the one or more ECG segments are noisy at step 912. In implementations, the remote server 102 may determine that the one or more ECG segments are noisy using the example process described above with respect to step 310 of FIG. 3 and FIG. 4. In this example, the remote server 102 determines that the score output of the machine learning classifier for the one or more ECG segments does not transgress a threshold; thus, the remote server 102 classifies the one or more ECG segments as noisy.

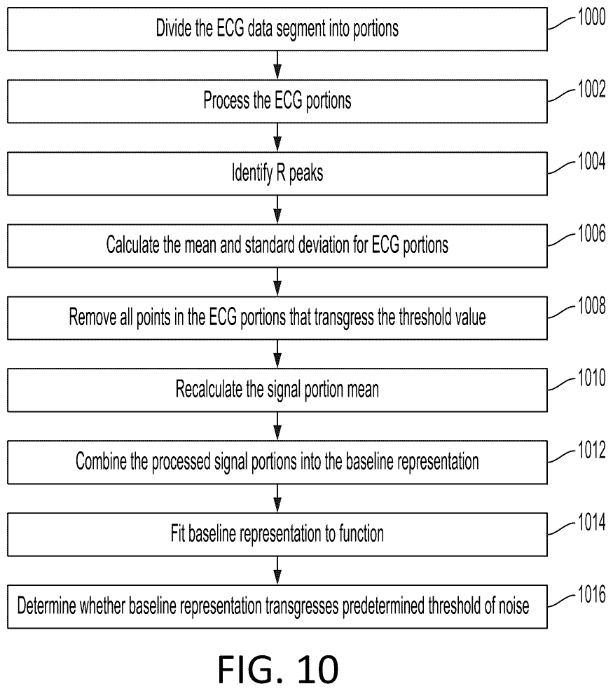

In implementations, the remote server 102 determines that the one or more ECG segments are noisy through the sample process flow illustrated in FIG. 10. The sample process of FIG. 10 can be implemented by the remote server 102 (e.g., at least one processor of the remote server 102. The remote server divides the ECG data of the one or more ECG segments into portions at step 1000. As an illustration, the remote server 102 may divide the ECG data into portions having a certain time period. For example, the remote server 102 may divide the ECG data segment into a number of subsample portions having a time period spanning between about 2 seconds and about 8 seconds. As another example, the remote server 102 may divide the ECG data segment into a number of subsample portions having a time period spanning 4 second portions. In implementations, the remote server 102 may not perform this step, as the one or more ECG segments have already been segmented by the cardiac device 100.

The remote server 102 processes the ECG portions at step 1002. Processing the ECG portions may include, for instance, filtering the ECG portions to remote ECG data contained within the portions that transgresses a particular threshold value. For example, for each portion the remote server 102 may identify the R-peaks in QRS complexes of the ECG portions at step 1004. For each R-peak, the remote server 102 can calculate the mean and standard deviation including the R-peaks at step 1006. As an illustration, the remote server 102 may determine a mean for the ECG data for each of the portions, determine the standard deviation of the ECG data for each of the portions, and calculate the threshold value based upon the mean and standard deviation. The threshold value can be calculated such that any portion of the ECG data identified as outliers when compared to the mean and standard deviation are excluded from the ECG data. Although means and standard deviations are described herein, other statistical measures can be used to make such calculations. For example, instead of the mean, a median or mode or other representative value for the various ECG data points may be used.

The remote server 102 may remove all points of the ECG portions that transgress the threshold at step 1008. For example, the remote server 102 may be configured to remote all points lying more than 2 standard deviations from the mean, thereby effectively removing the R-peaks from the ECG segment portions. Once the points have been removed, the remote server 102 may recalculate the mean amplitude for the portion at step 1010. The remote server 102 may then combine the points representing the mean amplitudes for the processed portion as the baseline representation at step 1012. Although this process is described as removing only R-peaks, the process may be adapted to additionally, or alternatively, remove other fiducial ECG peaks such as P-peaks, T-peaks, and/or U-peaks using a similar threshold determination process as described above for R-peaks.

The remote server 102 may fit the baseline representation to a function at step 1014. In implementations, the function may include at least one coefficient. For example, the remote server 102 can fit the baseline representation to a polynomial function, such as a third-degree polynomial function. As a further example, the resulting points representing a mean amplitude in each portion may be fit using a linear regression to a third-degree polynomial. In such an example, the coefficient can be a coefficient of a first-degree monomial of the third-degree polynomial. A polynomial function is shown by way of example. Alternatively, or in addition, other functions can be used. For example, a logarithm, exponential, hyperbolic, power, or trigonometric function can be used alone or in combination with a polynomial function.

The remote server 102 determines whether the baseline representation (or representations) transgresses a predetermined threshold of noise at step 1016. As an illustration, the remote server 102 may have a stored template baseline representation for the patient 104 (e.g., constructed from an ECG signal taken from the patient 104 during a setup process). The remote server 102 may then determine whether the baseline representation or representations created from the one or more ECG segments include a drift of at least the predetermined threshold from the template baseline representation for the patient 104. For example, if the data points for the ECG segment baseline representation differ from the template baseline representation on average by at least a predetermined percentage, the cardiac device 100 may determine that the ECG segment is noisy. The predetermined threshold of noise may be, for instance, 10%, 15%, 20%, 25%, 30%, 35%, 40%, 45%, 50%, etc. from the template baseline representation. In implementations, the predetermined threshold of noise may depend on a type of suspected arrhythmia corresponding to the one or more ECG segments. As an example, the predetermined threshold may be lower if the cardiac device 100 indicates to the remote server 102 that the patient 104 is experiencing ventricular fibrillation than if the cardiac device 100 indicates to the remote server 102 that the patient 104 is experiencing atrial fibrillation.

The remote server 102 is configured to transmit a request to the cardiac device 100 for additional physiological data for the patient 104 upon determining that the one or more ECG segments are noisy at step 914. For example, the remote server 102 may transmit the request for additional physiological data to the cardiac device 100 using MQTT protocols. The cardiac device 100 receives the request for additional physiological data at step 916. The cardiac device 100 then packages the additional physiological data for the remote server 102 at step 918. In implementations, the cardiac device 100 may package the additional physiological data for the remote server 102 similarly to the processes described above with reference to step 306 of FIG. 3.

In implementations, the additional physiological data for the patient 104 may include one or more additional ECG segments. For example, the cardiac device 100 may transmit one or more additional ECG segments from a different channel of the ECG electrodes 202 (or with a larger proportion of ECG segments from a second channel of the ECG electrodes 202 compared to a first channel of the ECG electrodes 202). As another example, the cardiac device 100 may transmit one or more additional ECG segments from a different time period (e.g., more current ECG segment(s)). In implementations, the cardiac device 100 may transmit non-ECG data to the remote server 102. As an example, the cardiac device 100 may transmit motion data (e.g., taken from an accelerometer or other motion sensor on the cardiac device 100). As another example, the cardiac device 100 may transmit vibrational data, such as cardiac vibrational data (e.g., vibrational data corresponding to heart sounds of the patient 104) and/or ambient vibrational data (e.g., vibrational data corresponding to the environment of the patient 104).

In implementations, packaging the additional physiological data includes identifying the physiological data to send to the remote server 102. For instance, the physiological data may be default data sent to the remote server 102 in response for a request for additional physiological data (e.g., the cardiac device 100 automatically sends the remote server 102 one or more ECG segments from a different ECG channel). In another example, the remote server 102 may identify a likely type of noise in the one or more ECG segments. To illustrate, the remote server 102 may identify that a signal amplitude in the one or more ECG segments is below a predetermined threshold and thus ECG data from another ECG channel should be used. As another illustration, the remote server 102 may identify that the noise in the one or more ECG segments is consistent with noise from one or more templates, such as a template for noise caused by movement, such that the remote server 102 may request motion data from the cardiac device 100. Accordingly, the cardiac device 100 may identify and package data according to the request from the remote server 102. Alternatively, or additionally, the cardiac device 100 may analyze a type of noise in the one or more ECG segments to determine what additional physiological data to transmit to the remote server 102.

Once the additional physiological data has been packaged, the cardiac device 100 transmits the packaged additional physiological data to the remote server 102 at step 920. In implementations, the cardiac device 100 transmits the packaged additional physiological data to the remote server 102 using similar processes to those described above with reference to step 306 of FIG. 3.

Steps 922 and 924 illustrate examples of actions the remote server 102 performs in response to the additional physiological data. In implementations, the remote server 102 receives the additional physiological data at step 922. The remote server 102 analyzes the one or more ECG segments and/or the received additional physiological data to determine whether the patient 104 is experiencing a treatable and/or alertable arrhythmia at step 924. As an example, the additional physiological data may include more current ECG segment(s) and/or ECG segment(s) from another channel of the ECG electrodes 202. The remote server 102 may then analyze these new ECG segment(s) using the processes described above with reference to step 310 of FIG. 3 to determine whether the patient 104 is experiencing a treatable and/or alertable arrhythmia. As another example, the additional physiological data may include motion data for the patient 104. The remote server 102 may analyze the motion data for the patient 104 to determine if the patient 104 is actively moving (e.g., if the motion data is from an accelerometer, by determining accelerometer counts over a certain time period from the motion data). If the patient 104 is actively moving, the remote server 102 may determine that the patient 104 is not likely experiencing a treatable and/or alertable arrhythmia. Alternatively, if the patient 104 is actively moving, the remote server 102 may modify one or more weights used to determine a confidence level for whether the patient 104 is experiencing a treatable and/or alertable arrhythmia to decrease the confidence level. As another example, the additional physiological data may include vibrational data for the patient 104. The remote server 102 may analyze the vibrational data to determine if, for instance, the patient 104 is moving and/or the patient 104 is in a noisy environment that could be causing the noise in the one or more ECG segments. If either or both of these appear to be true from the vibrational data, the remote server 102 may then accordingly determine that the patient 104 is not likely experiencing a treatable and/or alertable arrhythmia or modify one or more weights for a confidence level for whether the patient 104 is experiencing a treatable and/or alertable arrhythmia, as described above.

Based on the analysis of the one or more ECG segments and/or additional physiological data, the remote server 102 may generate a remote indication as to whether the patient 104 is experiencing a treatable and/or alertable arrhythmia. The remote server 102 may then transmit the remote indication to the cardiac device 100. In implementations, the remote server 102 may, alternatively or additionally, transmit the remote indication to a technician interface 106 associated with a technician and/or a caregiver interface 108 associated with a caregiver for the patient 104, as described above with reference to step 312 of FIG. 3.

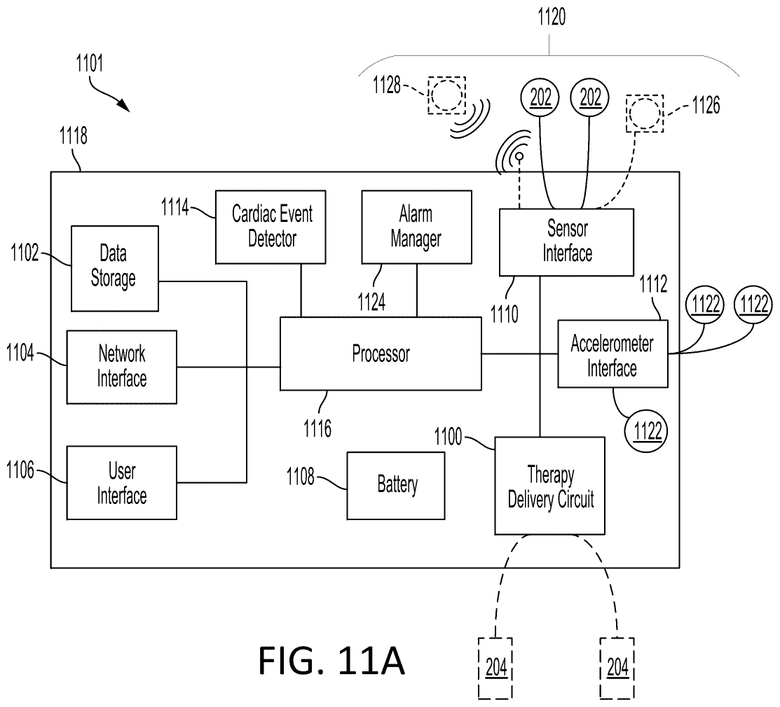

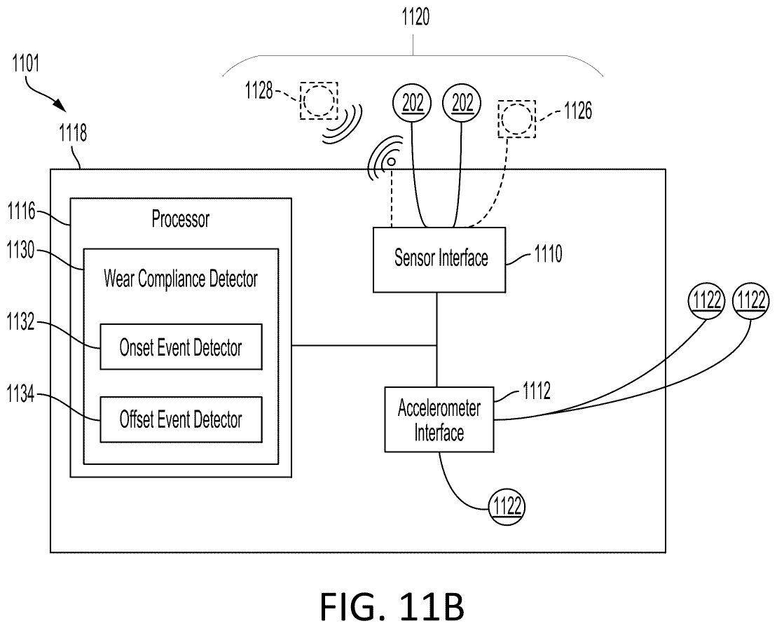

Returning to the wearable cardiac monitoring and treatment device 100, FIG. 11A illustrates a sample component-level view of a medical device controller 1101 included in a cardiac device 100. The medical device controller 1101 is an example of the medical device controller 206 shown in FIG. 2 and described above. As shown in FIG. 11A, the medical device controller 1101 can include a housing 1118 configured to house a therapy delivery circuit 1100 configured to provide one or more therapeutic shocks to the patient 104 via the therapy electrodes 204, a data storage 1102, a network interface 1104, a user interface 1106, at least one battery 1108 (e.g., within a battery chamber configured for such purpose), a sensor interface 1110 (e.g., to interface with the ECG electrodes 202 and other physiological sensors or detectors such as vibrational sensors, lung fluid sensors, infrared and near-infrared-based pulse oxygen sensors, and blood pressure sensors, among others), a cardiac event detector 1114, an alarm manager 1124, and at least one processor 1116. As described above, in some implementations, the cardiac device 100 that includes like components as those described above but does not include the therapy delivery circuit 1100 and the therapy electrodes 204 (shown in dotted lines). That is, in some implementations, the cardiac device 100 can include the ECG monitoring components and not provide therapy to the patient.

The therapy delivery circuit 1100 can be coupled to the therapy electrodes 204 configured to provide therapy to the patient 104. For example, the therapy delivery circuit 1100 can include, or be operably connected to, circuitry components that are configured to generate and provide an electrical therapeutic shock. The circuitry components can include, for example, resistors, capacitors, relays and/or switches, electrical bridges such as an h-bridge (e.g., including a plurality of insulated gate bipolar transistors or IGBTs), voltage and/or current measuring components, and other similar circuitry components arranged and connected such that the circuitry components work in concert with the therapy delivery circuit 1100 and under the control of one or more processors (e.g., processor 1116) to provide, for example, one or more pacing, defibrillation, or cardioversion therapeutic pulses. In implementations, pacing pulses can be used to treat cardiac arrhythmias such as bradycardia (e.g., less than 30 beats per minute) and tachycardia (e.g., more than 150 beats per minute) using, for example, fixed rate pacing, demand pacing, anti-tachycardia pacing, and the like. Defibrillation or cardioversion pulses can be used to treat ventricular tachycardia and/or ventricular fibrillation.

The capacitors can include a parallel-connected capacitor bank consisting of a plurality of capacitors (e.g., two, three, four, or more capacitors). In some examples, the capacitors can include a single film or electrolytic capacitor as a series connected device including a bank of the same capacitors. These capacitors can be switched into a series connection during discharge for a defibrillation pulse. For example, four capacitors of approximately 140 uF or larger, or four capacitors of approximately 650 uF can be used. The capacitors can have a 1600 VDC or higher rating for a single capacitor, or a surge rating between approximately 350 to 500 VDC for paralleled capacitors and can be charged in approximately 15 to 30 seconds from a battery pack.

For example, each defibrillation pulse can deliver between 60 to 180 J of energy. In some implementations, the defibrillating pulse can be a biphasic truncated exponential waveform, whereby the signal can switch between a positive and a negative portion (e.g., charge directions). This type of waveform can be effective at defibrillating patients at lower energy levels when compared to other types of defibrillation pulses (e.g., such as monophasic pulses). For example, an amplitude and a width of the two phases of the energy waveform can be automatically adjusted to deliver a precise energy amount (e.g., 150 J) regardless of the patient's body impedance. The therapy delivery circuit 1100 can be configured to perform the switching and pulse delivery operations, e.g., under control of the processor 1116. As the energy is delivered to the patient 104, the amount of energy being delivered can be tracked. For example, the amount of energy can be kept to a predetermined constant value even as the pulse waveform is dynamically controlled based on factors, such as the patient's body impedance, while the pulse is being delivered.

In certain examples, the therapy delivery circuit 1100 can be configured to deliver a set of cardioversion pulses to correct, for example, an improperly beating heart. When compared to defibrillation as described above, cardioversion typically includes a less powerful shock that is delivered at a certain frequency to mimic a heart's normal rhythm.

The data storage 1102 can include one or more of non-transitory computer readable media, such as flash memory, solid state memory, magnetic memory, optical memory, cache memory, combinations thereof, and others. The data storage 1102 can be configured to store executable instructions and data used for operation of the medical device controller 1101. In some implementations, the data storage 1102 can include sequences of executable instructions that, when executed, are configured to cause the processor 1116 to perform one or more functions. For example, the data storage 1102 can be configured to store information such as ECG data as received from, for instance, the sensor interface 1110.

In some examples, the network interface 1104 can facilitate the communication of information between the medical device controller 206 and one or more devices or entities over a communications network. For example, the network interface 1104 can be configured to communicate with the remote server 102 or other similar computing device. The network interface 1104 can include communications circuitry for transmitting data in accordance with a Bluetooth® wireless standard for exchanging such data over short distances to an intermediary device(s) (e.g., a base station, “hotspot” device, smartphone, tablet, portable computing device, and/or other device in proximity with the cardiac device 100). The intermediary device(s) may in turn communicate the data to the remote server 102 over a broadband cellular network communications link. The communications link may implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the intermediary device(s) may communicate with the remote server 102 over a Wi-Fi communications link based on the IEEE 802.11 standard. In some implementations, the network interface 1104 may be configured to instead communicate directly with the remote server 102 without the use of intermediary device(s). In such implementations, the network interface 1104 may use any of the communications links and/or protocols provided above.

In some implementations, the user interface 1106 may include one or more physical interface devices, such as input devices, output devices, and combination input/output devices, and a software stack configured to drive operation of the devices. These user interface elements may render visual, audio, and/or tactile content. Thus, the user interface 1106 may receive inputs and/or provide outputs, thereby enabling a user to interact with the medical device controller 206. In some implementations, the user interface 1106 may include a personal user device associated with the patient 104. In some implementations, the user interface 1106 may include a different device separate from the user interface 1106.

The medical device controller 206 can also include at least one battery 1108 configured to provide power to one or more components integrated in the medical device controller 206. The battery 1108 can include a rechargeable multi-cell battery pack. In one example implementation, the battery 1108 can include three or more cells (e.g., 2200 mA lithium ion cells) that provide electrical power to the other device components within the medical device controller 206. For example, the battery 1108 can provide its power output in a range of between 20 mA to 1000 mA (e.g., 40 mA) output and can support 24 hours, 48 hours, 72 hours, or more, of runtime between charges. In certain implementations, the battery capacity, runtime, and type (e.g., lithium ion, nickel-cadmium, or nickel-metal hydride) can be changed to best fit the specific application of the medical device controller 206.

The sensor interface 1110 can include physiological signal circuitry that is coupled to one or more externally worn sensors configured to monitor one or more physiological parameters of the patient and output one or more physiological signals. As shown, the sensors may be coupled to the medical device controller 1101 via a wired or wireless connection. The sensors can include one or more ECG electrodes 202 (e.g., ECG electrodes) configured to output at least one ECG signal. In some implementations, the sensors can include conventional ECG sensing electrodes and/or digital sensing electrodes. The sensors can also include one or more non-ECG physiological sensors 1120 such as one or more vibration sensors 1126, tissue fluid monitors 1128 (e.g., based on ultra-wide band RF devices), one or more motion sensors (e.g., accelerometers, gyroscopes, and/or magnetometers), a temperature sensor, a pressure sensor, a P-wave sensor (e.g., a sensor configured to monitor and isolate P-waves within an ECG waveform), an oxygen saturation sensor (e.g., implemented through photoplethysmography, such as through light sources and light sensors configured to transmit light into the patient's body and receive transmitted and/or reflected light containing information about the patient's oxygen saturation), and so on.

The one or more vibration sensors 1126 can be configured to detect cardiac or pulmonary vibration information. For example, the vibration sensors 1126 can detect a patient's heart valve vibration information. For example, the vibration sensors 1126 can be configured to detect cardio-vibrational signal values including any one or all of S1, S2, S3, and S4. From these cardio-vibrational signal values or heart vibration values, certain heart vibration metrics may be calculated, including any one or more of electromechanical activation time (EMAT), average EMAT, percentage of EMAT (% EMAT), systolic dysfunction index (SDI), and left ventricular systolic time (LVST). The vibration sensors 1126 can also be configured to detect heart wall motion, for instance, by placement of the sensor in the region of the apical beat. The vibration sensors 1126 can include a vibrational sensor configured to detect vibrations from a patient's cardiac and pulmonary system and provide an output signal responsive to the detected vibrations of a targeted organ, for example, being able to detect vibrations generated in the trachea or lungs due to the flow of air during breathing. In certain implementations, additional physiological information can be determined from pulmonary-vibrational signals such as, for example, lung vibration characteristics based on sounds produced within the lungs (e.g., stridor, crackle, etc.). The vibration sensors 1126 can also include a multi-channel accelerometer, for example, a three-channel accelerometer configured to sense movement in each of three orthogonal axes such that patient movement/body position can be detected and correlated to detected cardio-vibrations information. The vibration sensors 1126 can transmit information descriptive of the cardio-vibrations information to the sensor interface 1110 for subsequent analysis.

The tissue fluid monitors 1128 can use RF based techniques to assess fluid levels and accumulation in a patient's body tissue. For example, the tissue fluid monitors 1128 can be configured to measure fluid content in the lungs, typically for diagnosis and follow-up of pulmonary edema or lung congestion in heart failure patients. The tissue fluid monitors 1128 can include one or more antennas configured to direct RF waves through a patient's tissue and measure output RF signals in response to the waves that have passed through the tissue. In certain implementations, the output RF signals include parameters indicative of a fluid level in the patient's tissue. The tissue fluid monitors 1128 can transmit information descriptive of the tissue fluid levels to the sensor interface 1110 for subsequent analysis.

The controller 1101 can further include a motion detector interface operably coupled to one or more motion detectors configured to generate motion data, for example, indicative of physical activity performed by the patient 104. Examples of a motion detector may include a 1-axis channel accelerometer, 2-axis channel accelerometer, 3-axis channel accelerometer, multi-axis channel accelerometer, gyroscope, magnetometer, ballistocardiograph, and the like. As an illustration, the motion data may include accelerometer counts indicative of physical activity, accelerometer counts indicative of respiration rate, and posture information for the patient 104. For instance, in some implementations, the controller 1101 can include an accelerometer interface 1112 operably coupled to one or more accelerometers 1122, as shown in FIG. 11A. Alternatively, in some implementations, the accelerometer interface 1112 may be incorporated into other components of the controller 1101. As an example, the accelerometer interface 1112 may be part of the sensor interface 1110, and the one or more accelerometers 1122 may be part of the non-ECG physiological sensors 1120.

The accelerometer interface 1112 is configured to receive one or more outputs from the accelerometers. The accelerometer interface 1112 can be further configured to condition the output signals by, for example, converting analog accelerometer signals to digital signals (if using an analog accelerometer), filtering the output signals, combining the output signals into a combined directional signal (e.g., combining each x-axis signal into a composite x-axis signal, combining each y-axis signal into a composite y-axis signal, and combining each z-axis signal into a composite z-axis signal). In some examples, the accelerometer interface 1112 can be configured to filter the signals using a high-pass or band-pass filter to isolate the acceleration of the patient due to movement from the component of the acceleration due to gravity.

Additionally, the accelerometer interface 1112 can configure the output for further processing. For example, the accelerometer interface 1112 can be configured to arrange the output of an individual accelerometer 1122 as a vector expressing the acceleration components of the x-axis, the y-axis, and the z-axis as received from each accelerometer. The accelerometer interface 1112 can be operably coupled to the processor 1116 and configured to transfer the output signals from the accelerometers 1122 to the processor for further processing and analysis.

The one or more accelerometers 1122 can be integrated into one or more components of the cardiac device 100. In some implementations, one or more motion detectors 1122 may be located in or near the ECG electrodes 202. In some implementations, the one or more motion detectors 1122 may be located elsewhere on the cardiac device 100. For example, a motion detector 1122 can be integrated into the controller 1101. In some examples, a motion detector 1122 can be integrated into one or more of a therapy electrode 204, an ECG electrode 202, the connection pod 208, and/or into other components of the cardiac device 100. In some examples, a motion detector 1122 can be integrated into an adhesive ECG sensing and/or therapy electrode patch.

As described above, the sensor interface 1110 and the accelerometer interface 1112 can be coupled to any one or combination of sensing electrodes/other sensors to receive patient data indicative of patient parameters. Once data from the sensors has been received by the sensor interface 1110 and/or the accelerometer interface 1112, the data can be directed by the processor 1116 to an appropriate component within the medical device controller 1101. For example, ECG signals collected by the ECG electrodes 202 may be transmitted to the sensor interface 1110, and the sensor interface 1110 can transmit the ECG signals to the processor 1116, which, in turn, relays the data to the cardiac event detector 1114. The sensor data can also be stored in the data storage 1102 and/or transmitted to the remote server 102 via the network interface 1104. For instance, the processor 1116 may transfer the ECG signals from the ECG electrodes 202 and the motion data from the one or more accelerometers 1122 to the remote server 102.

In implementations, the cardiac event detector 1114 can be configured to monitor the patient's ECG signal for an occurrence of a cardiac event such as an arrhythmia or other similar cardiac event. The cardiac event detector can be configured to operate in concert with the processor 1116 to execute one or more methods that process received ECG signals from, for example, the ECG electrodes 202 and determine the likelihood that a patient is experiencing a cardiac event, such as a treatable and/or alertable arrhythmia. The cardiac event detector 1114 can be implemented using hardware or a combination of hardware and software. For instance, in some examples, cardiac event detector 1114 can be implemented as a software component that is stored within the data storage 1102 and executed by the processor 1116. In this example, the instructions included in the cardiac event detector 1114 can cause the processor 1116 to perform one or more methods for analyzing a received ECG signal to determine whether an adverse cardiac event is occurring, such as a treatable and/or alertable arrhythmia. In other examples, the cardiac event detector 1114 can be an application-specific integrated circuit (ASIC) that is coupled to the processor 1116 and configured to monitor ECG signals for adverse cardiac event occurrences. Thus, examples of the cardiac event detector 1114 are not limited to a particular hardware or software implementation.

In response to the cardiac event detector 1114 determining that the patient 104 is experiencing a treatable and/or alertable arrhythmia, the processor 1116 is configured to deliver a cardioversion/defibrillation shock to the patient 104 via the therapy electrodes 204. In some implementations, the alarm manager 1124 can be configured to manage alarm profiles and notify one or more intended recipients of events, where an alarm profile includes a given event and the intended recipients who may have in interest in the given event. These intended recipients can include external entities, such as users (e.g., patients, physicians and other caregivers, a patient's loved one, monitoring personnel), as well as computer systems (e.g., monitoring systems or emergency response systems, which may be included in the remote server 102 or may be implemented as one or more separate systems). For example, when the processor 1116 determines using data from the ECG electrodes 202 that the patient is experiencing a treatable arrhythmia, the alarm manager 1124 may issue an alarm via the user interface 1106 that the patient is about to experience a defibrillating shock. The alarm may include auditory, tactile, and/or other types of alerts. In some implementations, the alerts may increase in intensity over time, such as increasing in pitch, increasing in volume, increasing in frequency, switching from a tactile alert to an auditory alert, and so on. Additionally, in some implementations, the alerts may inform the patient that the patient can abort the delivery of the defibrillating shock by interacting with the user interface 1106. For instance, the patient may be able to press a user response button or user response buttons on the user interface 1106, after which the alarm manager 1124 will cease issuing an alert and the medical device controller 206 will no longer prepare to deliver the defibrillating shock.

The alarm manager 1124 can be implemented using hardware or a combination of hardware and software. For instance, in some examples, the alarm manager 1124 can be implemented as a software component that is stored within the data storage 1102 and executed by the processor 1116. In this example, the instructions included in the alarm manager 1124 can cause the processor 1116 to configure alarm profiles and notify intended recipients using the alarm profiles. In other examples, the alarm manager 1124 can be an application-specific integrated circuit (ASIC) that is coupled to the processor 1116 and configured to manage alarm profiles and notify intended recipients using alarms specified within the alarm profiles. Thus, examples of the alarm manager 1124 are not limited to a particular hardware or software implementation.

In some implementations, the processor 1116 includes one or more processors (or one or more processor cores) that each are configured to perform a series of instructions that result in the manipulation of data and/or the control of the operation of the other components of the medical device controller 1101. In some implementations, when executing a specific process (e.g., cardiac monitoring), the processor 1116 can be configured to make specific logic-based determinations based on input data received. The processor 1116 may be further configured to provide one or more outputs that can be used to control or otherwise inform subsequent processing to be carried out by the processor 1116 and/or other processors or circuitry with which the processor 1116 is communicably coupled. Thus, the processor 1116 reacts to a specific input stimulus in a specific way and generates a corresponding output based on that input stimulus. In some example cases, the processor 1116 can proceed through a sequence of logical transitions in which various internal register states and/or other bit cell states internal or external to the processor 1116 may be set to logic high or logic low.

As referred to herein, the processor 1116 can be configured to execute a function where software is stored in a data store (e.g., the data storage 1102) coupled to the processor 1116, the software being configured to cause the processor 1116 to proceed through a sequence of various logic decisions that result in the function being executed. The various components that are described herein as being executable by the processor 1116 can be implemented in various forms of specialized hardware, software, or a combination thereof. For example, the processor 1116 can be a DSP such as a 24-bit DSP processor. As another example, the processor 1116 can be a multi-core processor, e.g., having two or more processing cores. As another example, the processor 1116 can be an ARM, such as a 32-bit ARM processor. The processor 1116 can execute an embedded operating system and further execute services provided by the operating system, where these services can be used for file system manipulation, display and audio generation, basic networking, firewalling, data encryption, communications, and/or the like.

As noted above, a wearable cardiac monitoring device, such as the cardiac device 100, can be designed to include a digital front-end where analog signals sensed by skin-contacting electrode surfaces of a set of digital sensing electrodes are converted to digital signals for processing. Typical ambulatory medical devices with analog front-end configurations use circuitry to accommodate a signal from a high source impedance from the sensing electrode (e.g., having an internal impedance range from approximately 100 Kiloohms to one or more Megaohms). This high source impedance signal is processed and transmitted to a monitoring device such as processor 1116 of the controller 1101 as described above for further processing. In certain implementations, the monitoring device, or another similar processor such as a microprocessor or another dedicated processor operably coupled to the sensing electrodes, can be configured to receive a common noise signal from each of the sensing electrodes, sum the common noise signals, invert the summed common noise signals and feed the inverted signal back into the patient as a driven ground using, for example, a driven right leg circuit to cancel out common mode signals.

The cardiac device 100 is configured for long-term and/or extended use or wear by, or attachment or connection to, a patient. For example, devices as described herein may be capable of being continuously used or continuously worn by, or attached or connected to a patient, without substantial interruption (e.g., up to 24 hours or beyond, such as for weeks, months, or even years). In some implementations, such devices may be removed for a period of time before use, wear, attachment, or connection to the patient is resumed. As an illustration, devices may be removed to change batteries, carry out technical service, update the device software or firmware, and/or to take a shower or engage in other activities, without departing from the scope of the examples described herein. Such substantially or nearly continuous use or wear as described herein may nonetheless be considered continuous use or wear. Additionally, the cardiac device 100 may be configured to transmit signals and data to the remote server 102 continuously. For example, the cardiac device 100 shown and described with respect to FIGS. 2 and 11A-11B above may be configured to transmit signals and data to the remote server 102 continuously.

Implementations of the present disclosure may include monitoring medical device wear compliance for the patient 104. More specifically, the wear compliance information includes an accurate overview of what portion or percentage of a certain time period the patient has worn the cardiac device 100 and how this compares to the expected wear for the patient 104 as prescribed, for example, by their clinician or other healthcare provider when being prescribed the cardiac device. FIG. 11B illustrates an example reduced component-level view of the medical device controller 1101 that includes the processor 1116 that is configured to monitor wear compliance information for the patient 104 as described herein. For example, the processor 1116 can include wear time circuitry, such as a wear compliance detector 1130 as shown in FIG. 11B. The wear compliance detector 1130 may be integrated into the processor 1116 as illustrated in FIG. 11B, or the wear compliance detector 1130 may be integrated as a separate processing component operably coupled to the processor 1116. The wear compliance detector 1130 can be implemented as a dedicated microprocessor and associated circuitry disposed on a printed circuit board (PCB) along with other components as described herein. The wear compliance detector 1130, when implemented in a dedicated microprocessor or integrated into the processor 1116, can be based on a series of processor-readable instructions configured to be executed by the dedicated microprocessor or processor 1116. For example, the instructions can be implemented in a programming language such as C, C++, assembly language, machine code, HDL, or VHDL. In examples, the dedicated microprocessor can be an Intel-based microprocessor such as an X86 microprocessor or a Motorola 68020 microprocessor, each of which can use a different set of binary codes and/or instructions for similar functions. In implementations, the dedicated microprocessor or processor 1116 can be configured to implement wear onset event detection and wear offset event detection as set forth in FIG. 2B above.

As further shown in FIG. 11B, the wear compliance detector 1130 can include an onset event detector 1132 and an offset event detector 1134. As described above, the wear compliance detector 1130 can be a dedicated microprocessor and associated circuitry disposed on a PCB along with other components as described herein. In implementations, a first microprocessor can be implemented as the onset event detector 1132, and a second microprocessor can be implemented as the offset event detector 1134. In some implementations, both the onset event detector 1132 and offset event detector 1134 can be implemented in the same microprocessor as described above. The onset event detector 1132 and/or offset event detector 1134, when implemented in a dedicated microprocessor or integrated into the processor 1116, can be based on a series of processor-readable instructions configured to be executed by the dedicated microprocessor or processor 1116.

As noted above, when a patient puts on the cardiac device 100, a wear onset event can be determined based upon analysis of signals received from one or more of the sensors described herein. For example, based upon monitoring of signals output by the ECG electrodes 202 as well as signals output by the accelerometers 1122, the onset event detector 1132 can determine an onset event indicative of the patient 104 putting on or otherwise wearing the cardiac device 100. Similarly, the offset event detector 1134 can determine an offset event indicative of the patient 104 turning off, removing, or otherwise stopping the cardiac device 100 from monitoring. Based upon the measured onset and offset events, the wear compliance detector 1130 and/or the processor 1116 can determine wear compliance information (e.g., wear time) for the patient 104. Example operations executed by the processor 1116, wear compliance detector 1130, and the onset event detector 1132 and the offset event detector 1134 are described in additional detail in the above discussion of FIG. 2B and in U.S. patent application Ser. No. 16/951,246, entitled “Systems and Methods of Monitoring Wear Compliance of a Patient Wearing an Ambulatory Medical Device,” filed on Nov. 18, 2020, which as noted above is hereby incorporated by reference in its entirety.

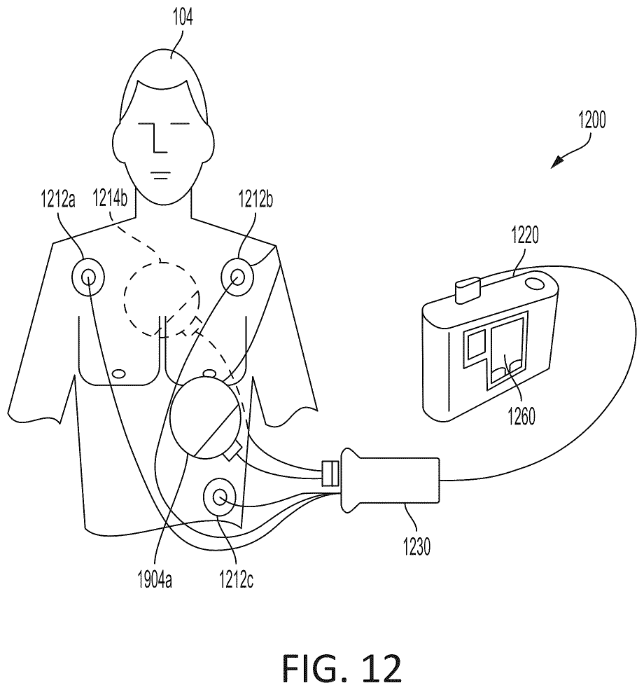

FIG. 12 illustrates another example of a wearable cardiac monitoring and treatment device 100. More specifically, FIG. 12 shows a hospital wearable defibrillator 1200 that is external, ambulatory, and wearable by the patient 104. Hospital wearable defibrillator 1200 can be configured in some implementations as a therapeutic device configured to provide pacing therapy, e.g., to treat bradycardia, tachycardia, and asystole conditions. The hospital wearable defibrillator 1200 can include one or more ECG sensing electrodes 1212a, 1212b, and 1212c (e.g., collectively ECG sensing electrodes 1212), one or more therapy electrodes 1214a and 1214b (e.g., collectively therapy electrodes 1214), a medical device controller 1220 and a connection pod 1230. For example, each of these components can be structured and function as similar components of the embodiments of the cardiac device 100 discussed above. In implementations, the electrodes 1212 and 1214 can include disposable adhesive electrodes. For example, the electrodes can include sensing and therapy components disposed on separate sensing and therapy electrode adhesive patches. In some implementations, both sensing and therapy components can be integrated and disposed on a same electrode adhesive patch that is then attached to the patient 104.

As an illustration, the front adhesively attachable therapy electrode 1214a attaches to the front of the patient's torso to deliver pacing or defibrillating therapy. Similarly, the back adhesively attachable therapy electrode 1214b attaches to the back of the patient's torso. In an example scenario, at least three ECG adhesively attachable sensing electrodes 1212 can be attached to at least above the patient's chest near the right arm (e.g., electrode 1212a), above the patient's chest near the left arm (e.g., electrode 1212b), and towards the bottom of the patient's chest (e.g., electrode 1212c) in a manner prescribed by a trained professional. In implementations, the example of the wearable cardiac monitoring device 100 shown in FIG. 12 may include additional adhesive therapy electrodes 1214 and/or the patches shown in FIG. 12 may include additional therapy electrodes 1214 on them such that at least two vectors may be formed between the therapy electrodes 1214 of the hospital wearable defibrillator 1200.

In implementations, the medical device controller 1220 may be configured to function similarly to the medical device controller 206 discussed above. As shown in FIG. 12, the medical device controller 1220 may include a user interface 1260 configured to communicate information with the patient 104. In examples, the patient 104 using the hospital wearable defibrillator 1200 shown in FIG. 19 may be monitored by a caregiver, such as hospital staff or a live-in or visiting caregiver. Additionally, in example, the hospital wearable defibrillator 1200 shown in FIG. 12 may need to be programmed for the patient 104. For example, a patient being monitored by a hospital wearable defibrillator and/or pacing device 1200 may be confined to a hospital bed or room for a significant amount of time (e.g., 75% or more of the patient's stay in the hospital). As a result, a user interface 1260 can be configured to interact with a user other than the patient, e.g., a nurse, for device-related functions such as initial device baselining, setting and adjusting patient parameters, and changing the device batteries.

In some implementations, an example of a therapeutic medical device that includes a digital front-end in accordance with the systems and methods described herein can include a short-term defibrillator and/or pacing device. For example, such a short-term device can be prescribed by a physician for patients presenting with syncope. A wearable defibrillator can be configured to monitor patients presenting with syncope by, e.g., analyzing the patient's physiological and cardiac activity for aberrant patterns that can indicate abnormal physiological function. For example, such aberrant patterns can occur prior to, during, or after the onset of syncope. In such an example implementation of the short-term wearable defibrillator, the electrode assembly can be adhesively attached to the patient's skin and have a similar configuration as the hospital wearable defibrillator 1200 described above in connection with FIG. 12.

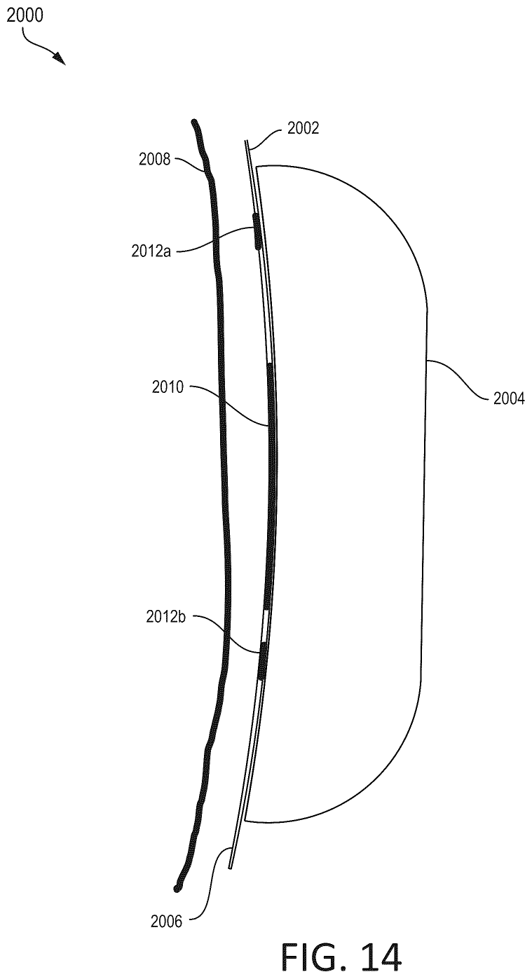

FIG. 14 illustrates another example of a wearable cardiac monitoring and treatment device 100. As shown in FIG. 14, the wearable cardiac monitoring device 100 may be or include an adhesive assembly 2000. The adhesive assembly 2000 includes a contoured pad 2002 and a housing 2004 configured to form a watertight seal with a contoured pad 2002. In implementations, the housing 2004 is configured to house electronic components of the adhesive assembly 2000, such as electronic components similar to components described above with respect to the embodiments of the wearable cardiac monitoring device 100 described above. The adhesive assembly 2000 includes a conductive adhesive layer 2006 configured to adhere the adhesive assembly 2000 to a skin surface 2008 of the patient 104. The adhesive layer 2006 may include, for example, a water-vapor permeable conductive adhesive material, such as a material selected from the group consisting of an electro-spun polyurethane adhesive, a polymerized microemulsion pressure sensitive adhesive, an organic conductive polymer, an organic semi-conductive conductive polymer, an organic conductive compound and a semi-conductive conductive compound, and combinations thereof.

The adhesive assembly 2000 also includes at least one therapy electrode 2010 integrated with the contoured pad 2002. In implementations, the adhesive assembly 2000 may include a therapy electrode 2010 that forms a vector with another therapy electrode disposed on another adhesive assembly 2000 adhered to the patient's body and/or with a separate therapy electrode adhered to the patient's body. The adhesive assembly 2000 may also include one or more ECG sensing electrodes 2012 integrated with the contoured pad 2002 (e.g., ECG sensing electrodes 2012a and 2012b). In implementations, the adhesive assembly 2000 may alternatively or additionally be in electronic communication with a separate ECG sensing electrode, such as an adhesive sensing electrode adhered to the patient's body. In examples, as shown in FIG. 20, the therapy electrode(s) 2010 and ECG sensing electrode(s) 2012 may be formed within the contoured pad 2002 such that a skin-contacting surface of each component is coplanar with or protrudes from the patient-contacting face of the contoured pad 2002. Examples of a wearable cardiac monitoring device 100 include an adhesive assembly 2000 are described in U.S. patent application Ser. No. 16/585,344, entitled “Adhesively Coupled Wearable Medical Device,” filed on Sep. 27, 2019, which is hereby incorporated by reference in its entirety.

Although the subject matter contained herein has been described in detail for the purpose of illustration, such detail is solely for that purpose and that the present disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

Other examples are within the scope and spirit of the description and claims. Additionally, certain functions described above can be implemented using software, hardware, firmware, hardwiring, or combinations of any of these. Features implementing functions can also be physically located at various positions, including being distributed such that portions of functions are implemented at different physical locations.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. Those skilled in the art will readily appreciate that all parameters, dimensions, materials, and configurations described herein are meant to be an example and that the actual parameters, dimensions, materials, and/or configurations will depend upon the specific application or applications for which the inventive teachings is/are used.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

1-63. (canceled)

64. A wearable cardiac monitoring and treatment system configured to remotely identify and/or verify treatable cardiac arrhythmias in an ambulatory patient, comprising:
a wearable cardiac monitoring and treatment device configured for long-term continuous cardiac monitoring of the ambulatory patient, the device comprising
a plurality of ECG electrodes configured to be in long-term continuous contact with skin of the ambulatory patient and sense electrical signals indicative of cardiac activity in the ambulatory patient,
a plurality of therapy electrodes configured to deliver one or more therapeutic electrical shocks to the ambulatory patient, and
a medical device controller coupled to the plurality of ECG electrodes and/or the plurality of therapy electrodes, and
a remote server in electronic communication with the medical device controller of the wearable cardiac monitoring and treatment device;
wherein the medical device controller comprises one or more processors configured to
generate an ECG signal based on the sensed electrical signals indicative of the cardiac activity in the ambulatory patient,
determine one or more ECG segments corresponding to a suspected arrhythmia occurring in the ambulatory patient based on the ECG signal,
transmit the determined one or more ECG segments corresponding to the suspected arrhythmia occurring in the ambulatory patient to the remote server,
receive, from the remote server, a remote indication based on the one or more ECG segments as to whether the ambulatory patient is experiencing a treatable arrhythmia,
independently analyze, at the medical device controller, the one or more ECG segments to generate a local indication as to whether the ambulatory patient is experiencing a treatable arrhythmia, and
determine whether to deliver, via the plurality of therapy electrodes, a therapeutic shock to the ambulatory patient based on the received remote indication and the local indication as to whether the ambulatory patient is experiencing a treatable arrhythmia.

65. The wearable cardiac monitoring and treatment system of claim 64, wherein the one or more processors are further configured to query the remote server for the remote indication upon generating a local indication that the ambulatory patient is experiencing a treatable arrhythmia.

66. The wearable cardiac monitoring and treatment system of claim 64, wherein the one or more processors are configured to determine whether to deliver the therapeutic shock to the ambulatory patient by determining whether the remote indication and the local indication indicate that the ambulatory patient is experiencing a treatable arrhythmia.

67. The wearable cardiac monitoring and treatment system of claim 66, wherein the one or more processors are further configured to delay the therapeutic shock to the ambulatory patient upon determining that the local indication indicates that the ambulatory patient is experiencing a treatable arrhythmia but that the remote indication does not indicate that the ambulatory patient is experiencing a treatable arrhythmia.

68. The wearable cardiac monitoring and treatment system of claim 67, wherein the one or more processors are further configured to perform a second analysis of the one or more ECG segments and/or of one or more additional ECG segments corresponding to the suspected arrhythmia to generate a second local indication as to whether the ambulatory patient is experiencing a treatable arrhythmia.

69. The wearable cardiac monitoring and treatment system of claim 68, wherein the one or more ECG segments are from a first channel of the plurality of ECG electrodes, and wherein the one or more additional ECG segments are from a second channel of the plurality of ECG electrodes.

70. The wearable cardiac monitoring and treatment system of claim 68, wherein the one or more ECG segments are from a first period of time, and wherein the one or more additional ECG segments are from a second period of time later than the first period of time.

71. The wearable cardiac monitoring and treatment system of claim 64, wherein the one or more processors are configured to determine the one or more ECG segments corresponding to the suspected arrhythmia occurring in the ambulatory patient based on the ECG signal using a first process; and
wherein the one or more processors are configured to independently analyze, at the medical device controller, the one or more ECG segments to generate a local indication as to whether the ambulatory patient is experiencing a treatable arrhythmia using a second process.

72. The wearable cardiac monitoring and treatment system of claim 71, wherein the first process comprises a lower power process than the second process.

73. The wearable cardiac monitoring and treatment system of claim 64, wherein the wearable cardiac monitoring and treatment device further comprises a garment configured to be worn about a torso of the ambulatory patient, wherein the plurality of ECG electrodes and the plurality of therapy electrodes are configured to be disposed on the garment.

74. A wearable cardiac monitoring and treatment system configured to remotely identify and/or verify treatable cardiac arrhythmias in an ambulatory patient, comprising:
a wearable cardiac monitoring and treatment device configured for long-term continuous cardiac monitoring of the ambulatory patient, the device comprising
a plurality of ECG electrodes configured to be in long-term continuous contact with skin of the ambulatory patient and sense electrical signals of the ambulatory patient indicative of cardiac activity in the ambulatory patient,
a plurality of therapy electrodes configured to deliver one or more therapeutic electrical shocks to the ambulatory patient, and
a medical device controller coupled to the plurality of ECG electrodes and/or the plurality of therapy electrodes, and
a remote server in electronic communication with the medical device controller of the wearable cardiac monitoring and treatment device;
wherein the medical device controller comprises one or more processors configured to
generate an ECG signal based on the sensed electrical signals indicative of the cardiac activity in the ambulatory patient,
determine one or more ECG segments corresponding to a suspected arrhythmia occurring in the ambulatory patient based on the ECG signal,
transmit the determined one or more ECG segments corresponding to the suspected arrhythmia occurring in the ambulatory patient to the remote server,
independently analyze, at the medical device controller, the one or more ECG segments to generate a local indication as to whether the ambulatory patient is experiencing a treatable arrhythmia,
upon generating a local indication that the ambulatory patient is experiencing a treatable arrhythmia, wait for a remote indication from the remote server as to whether the ambulatory patient is experiencing a treatable arrhythmia, and
after waiting a predetermined amount of time and receiving no remote indication as to whether the ambulatory patient is experiencing a treatable arrhythmia, determine whether to deliver, via the plurality of therapy electrodes, a therapeutic shock to the ambulatory patient.

75. The wearable cardiac monitoring and treatment system of claim 74, wherein the one or more processors are further configured to query the remote server for the remote indication upon generating a local indication that the ambulatory patient is experiencing a treatable arrhythmia.

76. The wearable cardiac monitoring and treatment system of claim 74, wherein the one or more processors are configured to determine whether to deliver the therapeutic shock to the patient by
performing a second independent analysis, at the medical device controller, of the one or more ECG segments and/or one or more second ECG segments to verify whether the patient is experiencing a treatable arrhythmia; and
generating a second local indication as to whether the ambulatory patient is experiencing a treatable arrhythmia.

77. The wearable cardiac monitoring and treatment system of claim 76, wherein the one or more ECG segments are from a first channel of the plurality of ECG electrodes, and wherein the one or more second ECG segments are from a second channel of the plurality of ECG electrodes.

78. The wearable cardiac monitoring and treatment system of claim 76, wherein the one or more ECG segments are from a first period of time, and wherein the one or more second ECG segments are from a second period of time later than the first period of time.

79. The wearable cardiac monitoring and treatment system of claim 76, wherein the one or more processors are further configured to initiate a treatment sequence upon generating a second local indication that the ambulatory patient is experiencing a treatable arrhythmia.

80. The wearable cardiac monitoring and treatment system of claim 76, wherein the one or more processors are further configured to determine whether to deliver the therapeutic shock to the patient by
upon generating a second local indication that the ambulatory patient is not experiencing a treatable arrhythmia, performing a third independent analysis, at the medical device controller, of the one or more ECG segments, the one or more second ECG segments, and/or one or more third ECG segments; and
generating a third local indication as to whether the ambulatory patient is experiencing a treatable arrhythmia.

81. The wearable cardiac monitoring and treatment system of claim 76, wherein the one or more processors are configured to determine the one or more ECG segments corresponding to the suspected arrhythmia occurring in the ambulatory patient based on the ECG signal using a first process; and
wherein the one or more processors are configured to independently analyze, at the medical device controller, the one or more ECG segments to generate a local indication as to whether the ambulatory patient is experiencing a treatable arrhythmia using a second process.

82. The wearable cardiac monitoring and treatment system of claim 81, wherein the first process comprises a lower power process than the second process.

83. The wearable cardiac monitoring and treatment system of claim 74, wherein the wearable cardiac monitoring and treatment device further comprises a garment configured to be worn about a torso of the ambulatory patient, wherein the plurality of ECG electrodes and the plurality of therapy electrodes are configured to be disposed on the garment.

84. A wearable cardiac monitoring and treatment system configured to remotely identify and/or verify treatable cardiac arrhythmias in an ambulatory patient, comprising:
a wearable cardiac monitoring and treatment device configured for long-term continuous cardiac monitoring of the ambulatory patient, the device comprising
a plurality of ECG electrodes configured to be in long-term continuous contact with skin of the ambulatory patient and sense electrical signals indicative of cardiac activity in the patient,
a plurality of therapy electrodes configured to deliver one or more therapeutic electrical shocks to the ambulatory patient, and
a medical device controller coupled to the plurality of ECG electrodes and/or the plurality of therapy electrodes, and
a remote server in electronic communication with the medical device controller of the wearable cardiac monitoring and treatment device, the remote server comprising one or more processors configured to
receive, from the wearable cardiac monitoring and treatment device, one or more ECG segments corresponding to a suspected arrhythmia occurring in the ambulatory patient based on the sensed electrical signals indicative of the cardiac activity in the patient from the wearable cardiac monitoring and treatment device,
analyze the received one or more ECG segments to determine whether the patient is experiencing a treatable arrhythmia,
determine that the one or more ECG segments are noisy, and
transmit a request to the wearable cardiac monitoring and treatment device for additional physiological data for the ambulatory patient upon determining that the one or more ECG segments are noisy.

85. The wearable cardiac monitoring and treatment system of claim 84, wherein the additional physiological data comprises one or more additional ECG segments.

86. The wearable cardiac monitoring and treatment system of claim 85, wherein the one or more ECG segments are from a first channel of the plurality of ECG electrodes, and wherein the one or more additional ECG segments are from a second channel of the plurality of ECG electrodes.

87. The wearable cardiac monitoring and treatment system of claim 85, wherein the one or more ECG segments are from a first period of time, and wherein the one or more additional ECG segments are from a second period of time later than the first period of time.

88. The wearable cardiac monitoring and treatment system of claim 84, wherein the additional physiological data comprises non-ECG data.

89. The wearable cardiac monitoring and treatment system of claim 88, wherein the additional physiological data comprises motion data.

90. The wearable cardiac monitoring and treatment system of claim 84, wherein the one or more processors are further configured to
receive the additional physiological data from the wearable cardiac monitoring and treatment device; and
analyze the received one or more ECG segments and/or received additional physiological data to determine whether the patient is experiencing a treatable arrhythmia.

91. The wearable cardiac monitoring and treatment system of claim 90, wherein the one or more processors are further configured to
generate a remote indication as to whether the patient is experiencing a treatable arrhythmia; and
transmit the remote indication to the wearable cardiac monitoring and treatment device.

92. The wearable cardiac monitoring and treatment system of claim 84, wherein the one or more processors are configured to determine that the one or more ECG segments is noisy by at least one of
determining that the one or more ECG segments transgress a predetermined threshold of noise; or
determining that the one or more ECG segments comprise a drift of at least the predetermined threshold of noise from a template baseline representation for the patient.

93. The wearable cardiac monitoring and treatment system of claim 84, wherein the wearable cardiac monitoring and treatment device further comprises a garment configured to be worn about a torso of the ambulatory patient, wherein the plurality of ECG electrodes and the plurality of therapy electrodes are configured to be disposed on the garment.
